(19) United States
(12) Patent Application Publication
(10) Pub. No.: US 2025/0107716 A1
(43) Pub. Date: Apr. 3, 2025

(54) Integrated Biomedical Systems and Methods for Health Monitoring and Coordinated Patient Care

(71) Applicant: Lexical AL, Inc., Irvine, CA (US)

(72) Inventor: Christian Matthew Holes, Deerfield Beach, FL (US)

(21) Appl. No.: 18/375,001

(22) Filed: Sep. 29, 2023

(51) Int. Cl. A61B 5/0205; A61B 5/00; A61B 5/021; A61B 5/11; A61B 5/1455; A61B 5/332

(52) U.S. Cl. CPC A61B 5/02055; A61B 5/002; A61B 5/0022; A61B 5/7267; A61B 5/7275; A61B 5/742; A61B 5/7475; A61B 5/021; A61B 5/1112; A61B 5/14552; A61B 5/332

(57) ABSTRACT

A platform for coordinated patient care is disclosed herein. The platform comprises at least one remote monitoring medical device with at least one sensor and processor for transmitting a patient's biological sensor data in real time. The patient's data may be accessed by a medical provider in real time. The present invention may be used for telehealth visits, short term acute treatment or for long term monitoring of chronic disease. The biological data that may be collected includes vital signs, such as the patient's heart rate, blood oxygen levels, blood pressure, and temperature. Methods for use of the medical device are also disclosed herein, the methods comprising real-time transmission and storage of a patient's data streams to a medical provider for the diagnosis and treatment of a medical condition. Methods disclosed herein may also include artificial intelligence and machine learning models to train on the patient's data to interpret immediate sensor readings and predict future medical outcomes for the patient.

### FIELD OF THE INVENTION

This invention relates generally to medical devices and processes of patient care utilizing wireless electronic communications. More specifically, this disclosure relates to methods and systems for sending, receiving, analyzing, and interpreting patient data, including data streams from a medical device in real-time.

### BACKGROUND

Advancements in technology have led to an increase in telehealth practice as many patients and practitioners prefer the convenience and efficiency of virtual visits. While in some cases there may be a benefit to visiting a medical provider in person, there are many cases in which an in-person visit is not necessary. Where a patient and a provider have a long-standing relationship, where a patient requires a refill of a routine prescription for a chronic condition, where mobility is difficult for a patient, or where a patient is immunocompromised and visiting a hospital can lead to exposure to harmful microorganisms, telehealth visits are indeed in the best interest of the patient.

One significant drawback to current telehealth practices is that the provider is often working with a more limited amount of information related to the patient's condition than if they were in an in-person visit. Generally, a practitioner does not have access to a patient's current vital signs or recent medical data during a telehealth visit. While some devices exist for obtaining a snapshot of some of the patient's vital signs, there is no comprehensive device for transmitting a patient's biological data in real-time to a provider, there are no mechanisms for a provider to control a biomedical device in real-time to obtain a patient's biological readings at the provider's discretion.

In addition to routine doctor's visits, there is a need for accurately monitoring and recording a patient's vital signs on an ongoing basis while away from medical facilities. Presently, technology for wearable medical devices is limited to data that is collected for a short period of time. In general, prior art methods and devices involve a micro controller for collecting a snapshot of one datapoint for the patient and transmitting that datapoint to a mobile device, such as a smartphone or tablet.

Another problem with current patient care practices is that patient data remains with each provider, which creates an obstacle to comprehensive patient care. Data that is maintained with each provider cannot be viewed easily by other providers and is not used for making medical decisions regarding patient care. In fact, providers are generally unaware that additional data may exist, which could affect decisions that the provider may make. While there are some providers who make an effort to share data, the resultant data is usually incomplete or difficult to understand. The issue is inherently related to where the data is created and resided. When data is owned by a provider, the data remains property of the provider and the patient is only allowed access to their data. The data cannot be accessed by other providers to gain a better understanding of the patient's overall health.

What is needed are comprehensive devices and methods of use that can provide a healthcare practitioner with real-time electronic medical records and/or patient data as well as accurate historical data for the diagnosis and treatment of a patient's medical condition.

The present invention solves this problem.

### SUMMARY OF THE INVENTION

In a preferred embodiment, the present invention discloses a coordinated patient care platform utilizing at least one medical device for the transmission of patient data in real time to a server for patient monitoring and care. This model of patient medical care can be best described as a Coordinated Care model, which achieves a higher standard of patient care through constant monitoring of a patient, real-time updates, and interoperable patient communications. Monitoring can include remote patient monitoring to single use observation for a telehealth visit. Aspects of that include recording human vital signs and including visualization of ear, nose and throat and heart and lung sounds. Also included are respiratory rate observation and an extended ability for monitoring cardiac output through a polyvinylidene difluoride (PVDF) sensor based seismocardiography and ballistocardiography module.

The medical devices have their own capabilities that achieve a coordinated care model along with the idea of a remote patient monitoring platform. To describe it, one might think of remote patient monitoring as one aspect of the device capability and coordinated care would be another aspect of the device capability. The device also comprises onboard artificial intelligence that has the ability to utilize large biological readings models on the device to assist with sensor readings and patient health predictions.

Coordinated care would use the device that constantly monitors or perhaps calling it “continuous monitoring” of a patient does it more justice. This, through the traditional means of “Continuity of Care,” would be achieved because more patient data is entered into the data record over a longer period of time. Trends, AI, treatment efficacy and outcomes could be easily observable for a patient across care providers. The platform provided herein functions as a centralized location for the patient's data to be stored. The consequence of maintaining the patient's data with the patient, rather than with each provider, is an improved method of storing and retrieving a patient's medical history, thereby improving the quality of care received by the patient.

Another novel aspect of the present invention is that the devices and the platforms do more than just remote patient monitor, they provide real time monitoring and data streaming and the ability to observe data in a large statistical data model, which provides equitable and democratized patient care model. i.e., if you will a “Coordinated and Automated Care Model” or “Coordinated and Automated Continuity of Care Model.”

For example, when monitoring a hypertensive patient with cognitive heart failure, data analyzed from this process and platform will provide a democratized ability to monitor and treat the patient more efficiently. Rather than waiting for periodic medical visits, the patient and provider may be notified of a potential issue as soon as the data is collected.

Another novel aspect of the present invention is how a provider can administer remote medical care. Much can be achieved without physically touching the patient.

The medical device(s) are a major part of that. They encompass multiple functions with a very well thought design and device onboard capabilities to not only perform biological medical readings but to also stream data in a way that is both real-time data transmission and delivered in innovative ways to the patient record, the medical record, and a caregiver and even 911 through the various described mechanisms we have gone over in our meetings and design flows.

It's this plus the platform and observations and other aspects that allow for the ultimate capability to observe, diagnose, and treat from a comprehensive medical monitoring and coordinated care platform.

Yet another novel aspect of the present invention is where the patient's medical data resides. Data resides with the patient, rather than with the provider, which causes gaps in care as a byproduct. Data belongs to the patient and is controlled by the patient rather than being controlled by the medical provider. Since the patient owns their own data, it is more easily shareable. By default, the patient's medical information is shared with the provider attending the patient once permission is granted to the provider. The present invention has the ability to granularly control patient data, which is accessible by the patient, resulting in the ability to share all of the information obtained from the device, data from patient interactions, and other medical record information. Moreover, artificial intelligence, such as the Large Biological Readings Model Analysis along with the present invention's alerting system, is then able to analyze and use the data in novel ways, such as providing health updates that can be communicated to a person in an easy-to-understand manner. For example, a patient can be notified of an urgent medical situation, and, through cognitive automation and artificial intelligence, a person can be notified automatically of the medical situation in an easy-to-understand manner, which explains to the patient what the situation means to them and what type of response is appropriate. As an example, a high AIC reading might result in a notification to the patient that the patient should eat healthier, stay on their medication, if they are taking any, and schedule a visit with their provider, if necessary. In another example, if the medical device detects a high blood pressure reading, it may notify the patient that their blood pressure is out of range and that the patient should seek attention from a medical professional. In summary, one novel aspect of the present invention is patient care through patient monitoring utilizing novel medical devices, integrated systems and methods and the guidance of AI and alerting modules.

A preferred embodiment of the present invention discloses a wearable remote monitoring tower for real-time collecting and recording of a patient's vital signs, including, but not limited to, the patient's temperature, oxygen levels, blood pressure, carbon dioxide levels, and pulse rate. Sensor module units may include pulse oximeter, heart rate sensor, blood pressure sensor, temperature sensor, electrocardiogramonitor, accelerometer, air pump, gyroscope, speaker, microphone, camera, seismocardiography, ballistocardiography, and GPS modules. Ports on the device allow for the extension of peripheral medical devices to connect to the present invention. For example, ECG wet electrodes and a fingertip pulse oximeter may be connected to the ports on the device, allowing the device to be used much like that of a large medical tower. This allows for medical readings to be obtained without patient intervention. The patient's data is viewable in real-time or may recorded for purposes of monitoring the patient's ongoing medical condition for chronic disease monitoring. Data points can be analyzed independently, or more than one data point can be triangulated to arrive at a diagnosis for a particular medical condition.

The patient's data can be analyzed by a medical professional as well as by an artificial intelligence (AI) application, which is programmed to train an analysis model on the patient's data in order to generate accurate predictions about the patient's medical condition. Machine learning models can also be integrated into the present invention for analysis, based on sensor data, in order to determine the likelihood of an emergency. For example, machine learning can be used for analysis of raw electrocardiogram (ECG) data to determine the likelihood of an arrhythmia. These models can be trained and improved as additional data is collected.

The patient's data is stored and available in real-time to a provider or caregiver for analysis prior to or during a telehealth visit. The present invention also discloses a video conferencing component for viewing the patient's data streams during telehealth visits in real-time or using a peer-to-peer connection. The patient and provider can access a secured website designed specifically to display data from the device disclosed herein and to meet virtually for review of the patient's data streams in real-time.

An auxiliary device for monitoring additional parameters, including, for example, an accelerometer, stethoscope, otoscope, gyroscope, chest straps, bio-stickers, or wet/dry electrodes to collect data for respiration, heart rhythm, or electrocardiogramay be coupled to the wearable remote monitoring tower without the need for a wireless internet connection. Chest straps and bio-stickers may also be coupled using Bluetooth technology for collecting data related to a patient's respiratory rate.

It is contemplated by the inventor that the present invention may collect and store data that could be collected on a patient monitoring tower during a hospital visit using the present invention. A preferred method disclosed herein includes remotely collecting and monitoring patient information that a nurse would be able to observe from a nurse's station.

The devices disclosed herein do not require the use of an additional computing device, such as a smartphone or tablet, for collecting or processing data. Data is obtained directly on the present invention and transmitted to a remote server for storage via wireless internet connection. In a preferred embodiment of the present invention, data is collected by the device disclosed herein. Data collection is not initiated by beginning a call with a medical provider. Instead, data is continuously collected and streamed live.

Data is securely stored and transmitted using mutual exchange certificates to verify authorization for accessing data. Device connection tokens may also be utilized to authenticate access to a patient's data. Patient data is stored with the patient's profile and requires that the patient grant access to providers or caregivers for access to their information. This workflow allows for all the patient's data to be stored in one place, rather than with individual providers.

By maintaining all patient records in one centralized location, providers and caregivers can obtain a more comprehensive record of the patient's data for care, including data from other providers and new patient medical information.

Also disclosed herein are methods of using the medical device disclosed herein for patient diagnosis and treatment. In one embodiment, a method for streaming acquired data from a medical device comprises transmitting, in real-time, the acquired data from a medical device to a network transmission server for bio-directional data transfer and messaging wherein the medical device is authenticated with a server and biological sensor data is streamed to a provider. Biomedical device sensors connect directly to the client services and can send and receive messages from the device to control the device remotely and receive data from the device in real-time.

Biological audio and video may also be sent to a live streaming receiving device, such as a provider's desktop computer, for a telehealth visit. A portion of the video stream may also be sent to VOD service for recording and access later.

In another embodiment, a method for streaming acquired data from a medical device is disclosed herein comprising transmitting, in real-time, the acquired data from a medical device to an IoMT server for storage. The data can be analyzed and trained on by artificial intelligence and machine learning models for generating predictions related to a patient's medical condition. Machine learning is utilized for analysis to learn what factors are important.

In this way, an artificial intelligence enabled telehealth platform may be provided that empowers a remote provider to perform artificial intelligence analysis on live streaming data. For example, real-time data such as biological sensor data acquired from a monitoring device may be streamed from a patient in one physical location to a healthcare provider at another physical location in a way that is efficient, secure, and that integrates effectively with existing healthcare systems. Further, by enabling the receiving device to initiate desired actions on the real time data, real-time provider assistance may be offered, which improves diagnostic efficiency and patient outcomes. For example, where the present invention detects an emergency in progress, methods disclosed herein may include contacting emergency services for immediate treatment of the patient without the need for human monitoring in real-time.

It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### DESCRIPTION OF THE DRAWINGS

The present disclosure will be better understood from reading the following description of non-limiting embodiments, with reference to the attached drawings, wherein below:

FIG. 1 shows a block diagram of a telehealth system according to an exemplary embodiment of the disclosure.

FIG. 2A shows a block diagram of a telehealth system that uses telehealth IoMT monitoring and real-time data transfer technologies.

FIG. 2B shows an example sequence diagram for establishing telehealth IoMT monitoring and real-time data transfer connection between a transmitting device and a receiving device of a telehealth system.

FIG. 2C shows an example sequence diagram for establishing a real-time peer-to-peer connection between a transmitting device capable of streaming biological audio or video and a receiving device of a telehealth system.

FIG. 2D an example sequence diagram for establishing a real-time peer-to-peer connection between a transmitting device capable of streaming biological audio, video, and/or data and a receiving device of a telehealth system further disclosing the technology used in establishing the peer-to-peer connection, biological media encoding, and parallel processing, which further describes streaming the biological media in real-time while saving and storing the biological media for retrieval on demand.

FIG. 2E shows an example sequence diagram for establishing peer-to-peer connection between a transmitting device capable of streaming biological audio, video, and/or data and a receiving device of a telehealth system, further describing a methodology of connecting a peer-to-peer system based on a standardized specification that describes the egress (WHEP) and ingress (WHIP) WebRTC protocols.

FIG. 2F shows an example block diagram for a biological chip architecture for a medical device capable of streaming biological audio, video, and/or data.

FIG. 3 is a high-level flowchart illustrating an example method for live streaming data using an IoMT data streaming server and/or peer-to-peer technology during a telehealth visit.

FIG. 4 is a block diagram for capturing and processing multiple biological data points for increasing the prediction of the infrared body temperature sensor accuracy using artificial intelligence.

FIG. 5 is a block diagram for capturing and processing multiple biological data points for the purpose of obtaining cardiac output data from non-invasive methods using biological sensors and artificial intelligence to increase accuracy of sensor predictions.

FIG. 6 is a block diagram for capturing and processing multiple biological data points for the purpose of comparing normal sinus rhythm to irregularly irregular arrhythmia from a single-lead ECG sensor using artificial intelligence to increase accuracy of sensor predictions.

FIG. 7 is a high-level flowchart illustrating an example method for a medical device that determines the occurrence of a medical event which requires initiating a data transmission of an alert to an entity assigned to receive the alert through a peer-to-peer connection and provides a notification or the initiation of a telehealth visit.

FIG. 8a is a block diagram of a system for collecting and storing patient data and information in a centralized location that encompasses multiple sources of patient information and patient data that is deposited into a patient data store which is owned and authorized for medical used by a patient for the purpose of providing care.

FIG. 8b is a block diagram of a system for collecting and storing patient data and information in a centralized location that encompasses multiple sources of patient information and patient data that is deposited into a patient data store which is owned and authorized for medical used by a patient for the purpose of providing care, further comprising an alerting rely system utilizing artificial intelligence (large biological readings model) that determines an emergent medical event based on the collective patient information and can transmit the alert to an authorized entity.

8c depicts a flowchart establishing parameters for a notification system and artificial intelligence analysis of patient data and information from one or more data sources further comprising a scoring system for providing weights of influence based on the assigned parameters of scoring metrics used to influence the artificial intelligence analysis.

FIG. 8d depicts a flowchart for providing an assessment to a patient that has a scoring mechanism that is used for artificial intelligence analysis. The assessment can be an existing medical assessment, such as a PHQ9 (PHQ2/7), SDOH, and/or one or more custom patient assessments for providing weights of influence based on the assigned parameters of scoring metrics used to influence the artificial intelligence analysis, which may or may not require a response.

FIG. 8e is a schematic diagram depicting a remote patient monitoring system capable of utilizing data, including data from provider encounters, medical device readings and interactions, medical assessments, and patient messages sent to provider for the purpose of analyzing the data through artificial intelligence and determining if there is a medical event such as an emergent health situation or a deterioration in their normal health pattern, which may require a response. Furthermore, a scoring metric for each component is applied based upon incoming data results and are applied to influence the weights of analysis by the artificial intelligence models.

FIG. 8f is a block diagram depicting an artificial intelligence system for analyzing a video or image from a biological audio and/or video enabled medical device. The medical device may be, for example, a digital otoscope. The medical device further comprises a mechanism, which trains-on medical images for predicting a medical finding based on the analysis of on-device video image recordings taken from a patient.

FIG. 8g is a block diagram depicting an artificial intelligence system for analyzing a medical audio recording, such heart and lung auscultation, from a biological audio and/or video enabled medical device. The medical device may be, for example, a digital stethoscope. The medical device further comprises a mechanism, which trains-on medical audio for predicting a medical finding based on the analysis of on-device audio recordings taken from a patient.

FIG. 8h is a schematic diagram depicting an artificial intelligence system utilizing human cognitive process automation that analyzes medical and behavioral health conditions of a patient for the purpose of interaction with that patient through conversational automation systems that utilize, but are not limited to, large language models and natural language processing. The system further comprising a method of monitoring and analyzing the human and cognitive automation interactions and further training and correcting the model through medical provider feedback mechanism.

FIG. 8i is a schematic representation of a medical device capable of setting reminders and selecting various modes of operation for the purpose of utilizing the medical device for remote patient monitoring.

FIG. 9 is a schematic representation of system capable of multiple patient observation through remote patient monitoring enabled medical devices, further comprising a mechanism to alert medical staff responders and/or emergency medical responders of a medical event requiring a response. The system also comprises algorithms and artificial intelligence to determine the occurrence of a medical emergency.

FIG. 10 discloses a flow diagram describing an enhanced 911 and alerting services protocol of a biomedical monitoring device that comprises a network transceiver and GPS module that is capable of detecting a patient medical event and alerting medical staff, caregivers, and/or enhanced 911 services of the medical event and the location of the patient, as required.

FIGS. 11A-G constitute several views of remote health monitoring device, in accordance with one embodiment.

FIGS. 12A-E constitute several views of a remote data collection device, in accordance with one embodiment of the present invention.

FIG. 13 discloses one embodiment of a biosticker disclosed herein comprising a piezo electric sensor, gyroscope, accelerometer, temperature sensor, and blue tooth network transceiver micro controller component.

FIG. 14 is an ECG data transmission directly from the remote health monitoring device disclosed herein to a receiving device.

FIG. 15 is a plot point graph showing actual versus predicted plot points over time, highlighting the model's accuracy core body temp prediction from a machine learning algorithm.

FIG. 16 discloses a variation of a Bland-Altman plot line, which shows the acceptable ranges of variables that are incorporated into the model. Where actual readings fall between the predicted range, the non-linear polynomial regression calculation is robust. It is used to determine the variables are used to predict within predicted range. predicted versus actual ran.

FIG. 17 This figure illustrates a plot of deleted (or externally) studentized residuals for predicted core body temperatures.

FIG. 18. discloses a histogram illustrating the frequency distribution of predicted core body temperatures by the model.

### DETAILED DESCRIPTION OF THE INVENTION

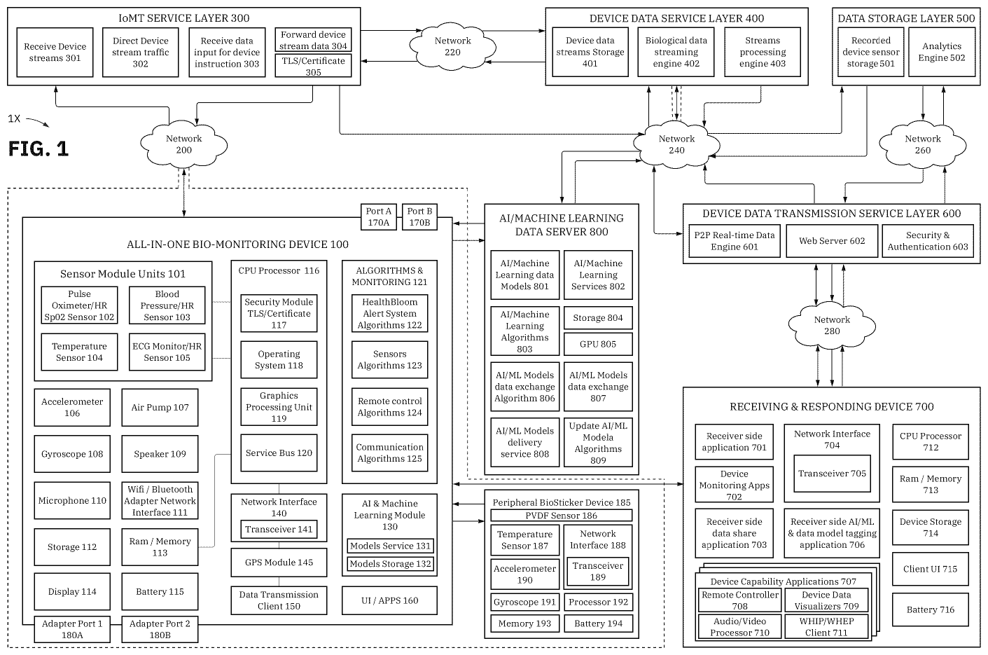

FIG. 1 shows a block diagram of a telehealth system according to an exemplary embodiment of the disclosure. The telehealth system comprises an all-in-one bio-monitoring device 100. The bio-monitoring device comprises sensor module units 101, a CPU processor 116, algorithms and monitoring 121, a machine learning artificial intelligence module 130, a network interface 140, a GPS module 145, data transmission client 150, a user interface 160, at least one port 170A and 170B, and at least one adapter 180A and 180B. The all-in-one bio-monitoring device also comprises an accelerometer 106, an air pump 107, a gyroscope 108, a speaker 109, a microphone 110, a Wi-Fi/Bluetooth network adapter interface 111, storage 112, Ram/memory 113, a display 114, and a battery 115. The sensor module units include a pulse oximeter/heartrate sensor 102, blood pressure/heartrate sensor 103, temperature sensor 104, and an ECG monitor/heartrate sensor 105. The CPU processor 116 comprises at least one security module TLS/Certificate 117, an operating system 118, a graphics processing unit 119, and a service bus 120. The algorithms and monitoring components 121 comprise at least a HealthBloom Alert System Monitoring algorithm 122, sensors algorithms 123, a remote-control algorithms 124, and communication algorithms 125. The network interface 140 comprises at least a network transceiver component 141. The artificial intelligence & machine learning module 130 comprises at least one artificial intelligence model service 131 and at least one artificial intelligence model for data storage 132.

The all-in-one bio-monitoring device communicates with a network 200 that connects with an IoMT service layer 300. The IoMT service layer 300 receives device streams 301, directs device stream traffic 302, receives data input for device instruction 303, forwards device stream data 304 and provides a server security TLS/Certificate 305. The IoMT service layer 300 communicates with a network 220, which connects to a device data service layer 400. The device data service layer 400 comprises receiving device data streams storage 401, biological data streaming engine 402, and a streams processing engine 403. The device data service layer 400 communicates with a network 240, which connects to a device data transmission service layer 600. The device data transmission service layer 600 transmits data through a network 260, which connects to a data storage service layer 500 and stores recorded device sensor data 501 and contains an analytics engine 502. The device data service layer 400 communicates with a network 240 which connects to a data storage service layer 500. This data storage service layer 500 comprises at least a recorded device sensor storage 501 and an analytics engine 502. The device data service layer 400 communicates with a network 240, which connects to a device data transmission service layer 600 that comprises a peer-to-peer real-time data engine 601, a web server 602 and security and authentication services 603. This device data transmission service layer 600 communicates with a network 280 that connects to a receiving and responding device 700. The receiving and responding device 700 comprises a receiver side application 701, a network interface 704 further comprising a network transceiver 705, receiver side artificial intelligence & machine learning, and data model tagging application 706, receiver side data share application 703, device monitoring applications 702, device capability applications 707, including remote controller 708, device data visualizers 709, real-time client video calling and messaging applications that may include WebRTC technologies such as WHIP/WHEP servers and clients 711, and audio/video processors 710. The receiving and responding device 700 also comprises a CPU processor 712, Ram/memory 713, device storage 714, client user interface 715 and a battery 716. The network 240 further communicates with a machine learning data server 800 which communicates with the all-in-one bio-monitoring device 100. The artificial intelligence & machine learning data server 800 comprises artificial intelligence & machine learning data models 801, artificial intelligence & machine learning algorithms 803, artificial intelligence & machine learning models data exchange algorithms 806, artificial intelligence & machine learning models delivery service 808, artificial intelligence & machine learning services 802, compute storage 804, GPU 805, artificial intelligence & machine learning models data exchange 807 and an artificial intelligence & machine learning update models algorithms 809.

The all-in-one bio-monitoring device 100 may also communicate with a peripheral bio-sticker device 185. The bio-sticker device 185 may comprise a polyvinylidene difluoride (PVDF) piezoelectric sensor 186, a temperature sensor 187, an accelerometer 190, a gyroscope 191, a compute RAM/memory component 193, a network interface 188, a network transceiver 189, a computer processing unit or micro-controller processor 192, and a battery component 194.

The all-in-one bio-monitoring device 100 may also communicate directly with a receiving and responding device 700.

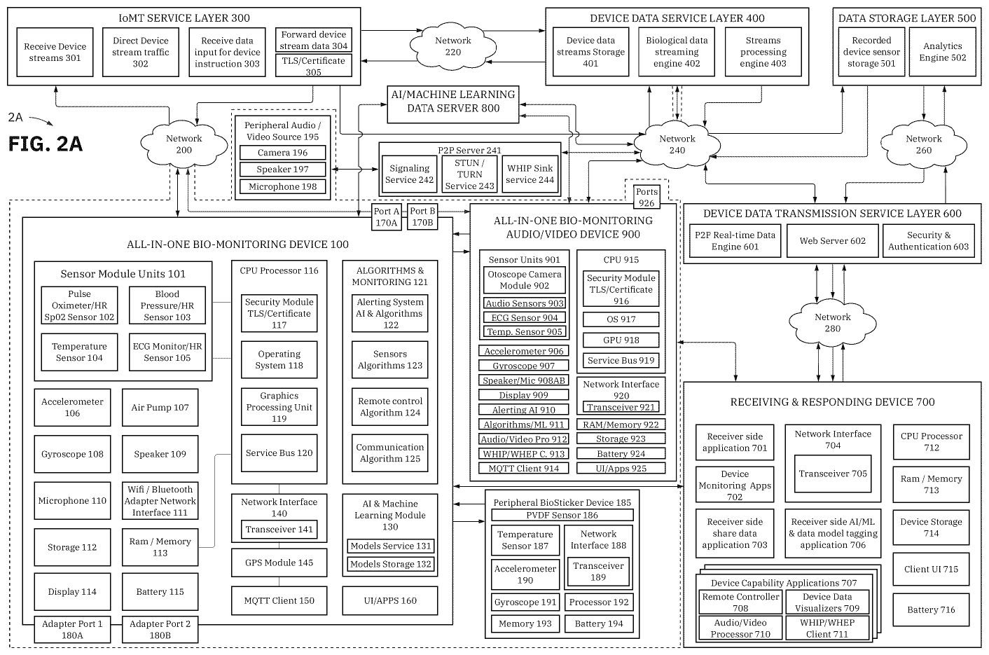

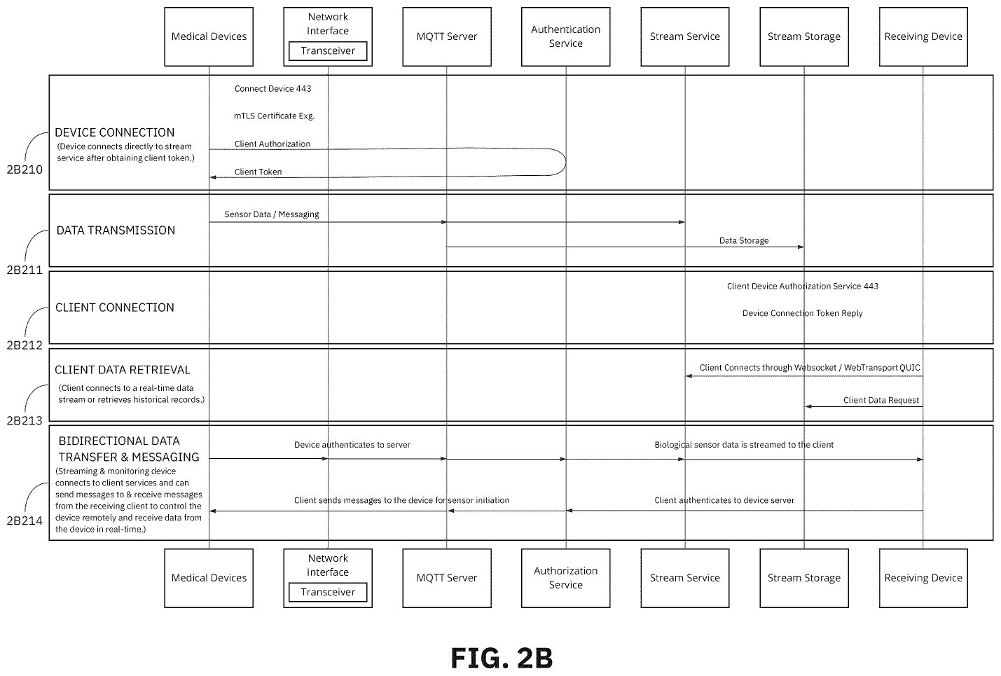

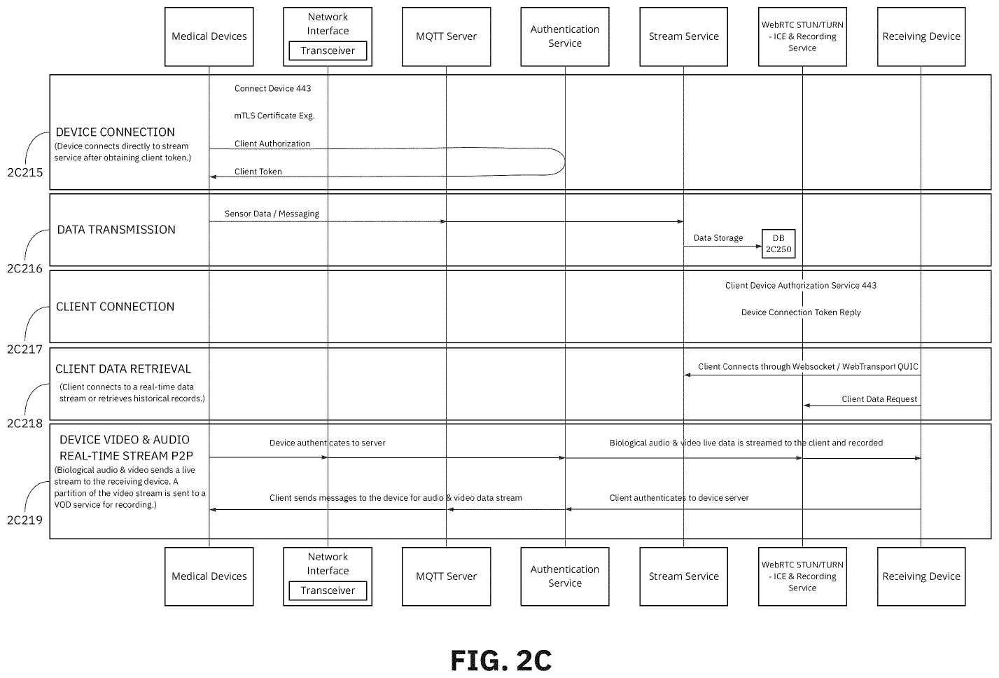

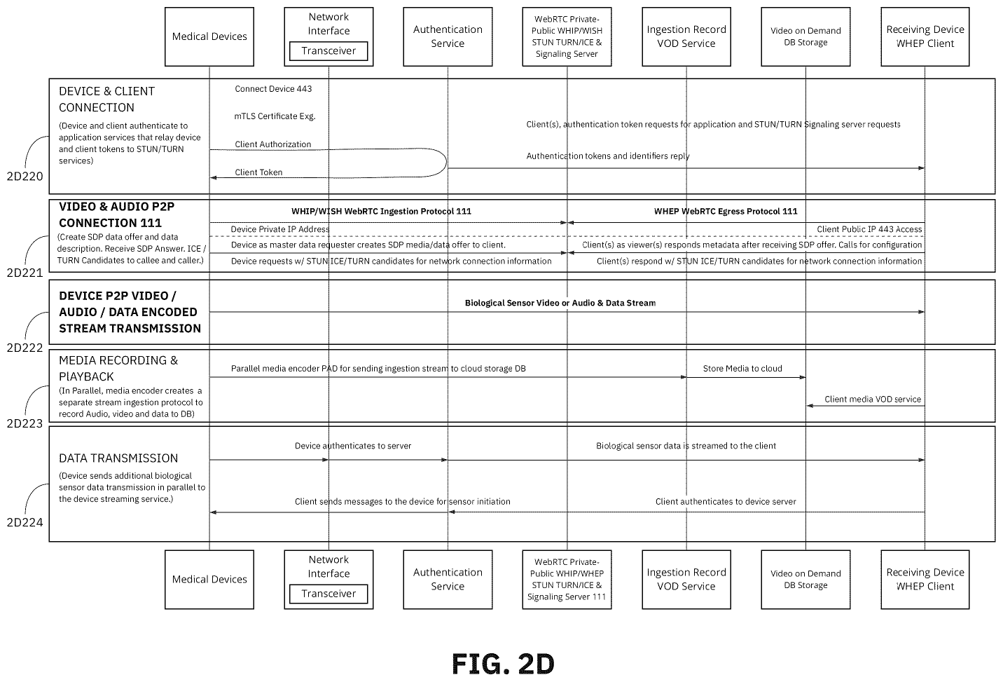

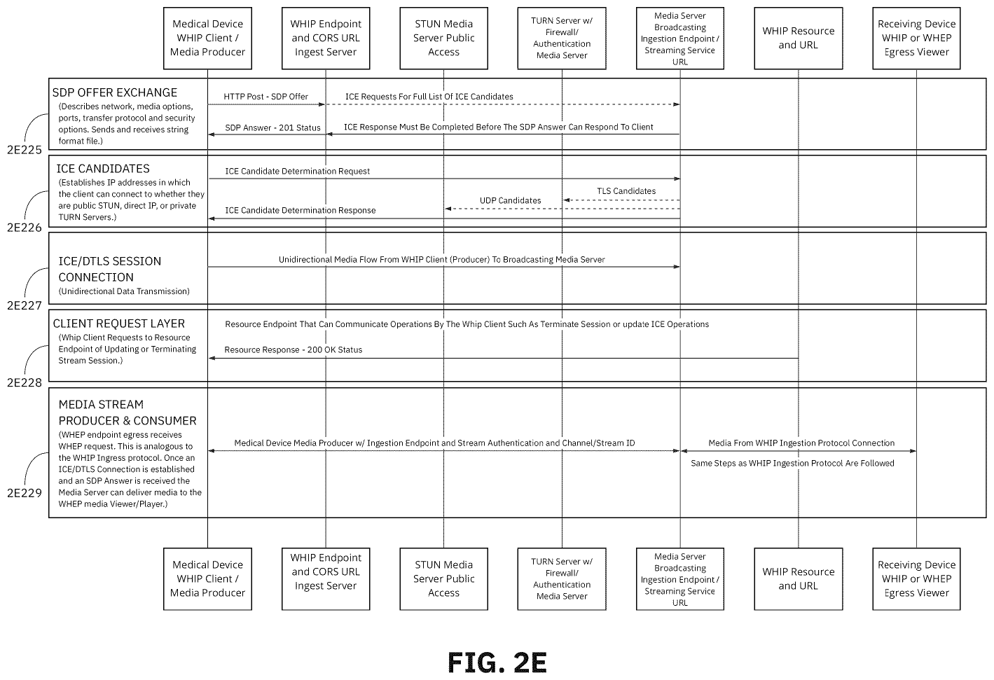

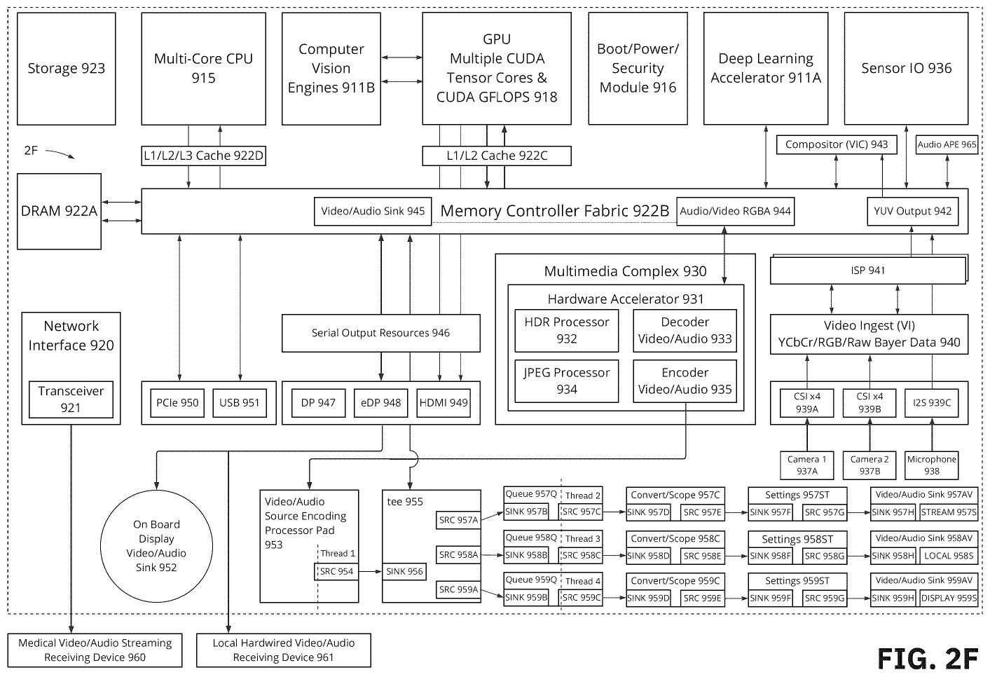

FIG. 2 shows a block diagram of a telehealth system according to an exemplary embodiment of the disclosure. The telehealth system comprises an all-in-one bio-monitoring device 100. The bio-monitoring device 100 comprises sensor module units 101, a CPU processor 116, algorithms and monitoring 121, an artificial intelligence & machine learning module 130, a network interface 140, a GPS module 145, IoMT client 150, a user interface and applications on the device 160, at least one port 170A, 170B, and at least one adapter 180A, 180B. The all-in-one bio-monitoring device 100 also comprises an accelerometer 106, an air pump 107, a gyroscope 108, a speaker 109, a microphone 110, a Wi-Fi/Bluetooth adapter network interface 111, storage, Ram/memory 113, a display 114, and a battery 115. The sensor module units 101 include a pulse oximeter/heartrate sensor 102, blood pressure/heartrate sensor 103, temperature sensor 104, and an ECG monitor/heartrate sensor 105. The all-in-one bio-monitoring device 100 connects with a network 200 that communicates with an MQTT IoMT service layer 300. The IoMT service layer 300 receives device streams 301, directs device stream data 302, receives data input for device instruction 303, forwards device stream data 304 and provides a TLS/Certificate 305 for device data to network security. The MQTT IoMT service layer 300 communicates with a network 220, which connects to a device data service layer 400. The device data service layer 400 comprising receiving device data streams storage 401, biological data streaming engine 402, and a streams processing engine 403. The device data service layer 400 communicates with a network 240, which connects to a data storage service layer 500. The data storage service layer 500 stores recorded device sensor data 501 and contains an analytics engine 502. The data storage service layer 500 communicates with a network 260, which connects the device data transmission service layer 600 that comprises a peer-to-peer real-time data engine 601, a web server 602 and security and authentication services 603. This service layer 600 communicates with a network 280 that connects to a receiving and responding device 700. The receiving and responding device 700 comprises a receiver side application 701, a network interface 704 further comprising a network transceiver 705, receiver side artificial intelligence & machine learning and data model tagging 706, receiver side data share application 703, device monitoring applications 702, device capability applications 707, including remote controller 708, device data visualizers 709, real-time client video calling and messaging applications that may include WebRTC technologies such as WHIP/WHEP servers and clients 711. The receiving and responding 700 device also comprises a CPU processor 712, Ram/memory 713, device storage 714, client user interface 715 and a battery 716. The network 280 and 240 further communicates with an artificial intelligence & machine learning data server 800 which communicates with the all-in-one bio-monitoring device 100 and an all-in-one bio-monitoring audio and video device 900. A peer-to-peer server 241 comprising a signaling service 242, STUN/TURN service 243, and WHIP/WHEP sink service 244 may also be connected to a network 240 for communication with other components of the present invention. The peer-to-peer server 241 further communicates with a peripheral audio/video source 190. The peripheral audio/video source 195 comprises at least a camera component 196, a speaker component 197, and a microphone component 198.

The networks 200 and 240 may also be connected to an all-in-one bio-monitoring video and/or audio medical device 900 comprising sensor units 901, a CPU processor 915, a network interface 920, further comprising a network transceiver 921, RAM/memory 922, storage 923, battery 924, and a user interface 925 and may have additional on-device software applications. The sensor units 901 may include an otoscope camera module 902, audio sensors 903, ECG sensors 904, temperature sensors 905. The medical device further comprising an accelerometer 906, a gyroscope 907, a speaker 908A, a microphone 908B, a display 909, alerting artificial intelligence 910, artificial intelligence & machine learning algorithms 911, audio processors 912A and video processors 912B, WHIP/WHEP real time communications client 913, and IoMT data transmission client 914. The medical device further comprises a central processing unit (CPU) 915, a security module with TLS/certificate embedded component 916, an operating system 917, a graphics processing unit (GPU) 918 and a service bus 919.

The all-in-one bio-monitoring medical device 100 may also communicate with a peripheral bio-sticker device 185. The bio-sticker device may comprise a polyvinylidene difluoride (PVDF) piezoelectric sensor 186, a temperature sensor 187, an accelerometer 190, a gyroscope module 191, a compute RAM/memory component 193, a network interface 188, a network transceiver 189, a computer processing unit or micro-controller processor 192, and a battery component 194.

FIG. 2B shows an example sequence diagram for establishing peer-to-peer connection between a transmitting device and a receiving device of the remote biological health monitoring system and telehealth real-time live stream system 2A of FIG. 2A. The diagram depicts device connection 2B210 from medical devices directly to a stream service after obtaining a client token. The medical device interfaces with a network using a transceiver to an IoMT server and connecting to a receiving device utilizing a mTLS certificate exchange. The medical device may also utilize an authentication service before connecting to a receiving device. Data transmission requires client authorization for transmission to a receiving device and a client token for communication back to the medical device.

Data transmissions 2B211 may transfer sensor data and/or messages from a medical device to an IoMT server, an authentication service, or a streaming service. As previously mentioned, when a biological monitoring device is connected to the network 200 described in the remote biological health monitoring system and telehealth real-time live stream system 2A of FIG. 2A, then a receiving device client connection 2B212 may authenticate with an authentication service, which may be part of the IoMT Service 300, across a network connection 200 secured by an SSL/TLS certificate 305 and receiving a device tokenized authentication key to establish a secure connection of the transmitting biomedical monitoring device to the receiving device. The receiving device utilizes client authorization services to authenticate and authorize the end user for the purpose of accessing patient data and messages from the broadcasting biomedical device.

The receiving device client may receive biological monitoring data through the client data retrieval stage 2B213 by either connecting to the data transmission in real-time or by retrieving historical data by requesting data that is permissioned to the client for retrieval. In a real-time data streaming connection scenario, the stream service may be alerted of a data connection and subsequent client data retrieval 2B213 through a WebSocket or WebTransport QUIC web client protocol. The client connection for receiving live data from the biomedical monitoring device may authenticate by receiving a device granted TLS certificate exchange as described earlier in the IoMT service 300 connection of the remote biological health monitoring system and telehealth real-time live stream system 2A of FIG. 2A. In another scenario, the end user may want to view historical data that they are authenticated and authorized to receive through a client data request.

Data and messages may be transferred bidirectionally 2B214. The streaming & monitoring device connects to client services may send messages to and receive messages from the receiving client device to control the device remotely and receive data from the device in real-time. Data may be sent between the medical device and the receiving device once the device has been authenticated with the server. Once the authorization service has authenticated the device, biological sensor data is streamed to the receiving device.

FIG. 2C shows an example sequence diagram for establishing peer-to-peer connection between a transmitting device and a receiving device described in the remote biological health monitoring system and telehealth real-time live stream system 2A of FIG. 2A. The medical device connects 2C215 directly to a stream service after obtaining a client token. The device connects to an IoMT server through a network transceiver on the device. The IoMT server initiates an mTLS certificate exchange to the medical device for communication of data broadcasting from the medical device.

Sensor data and/or messages may also be transmitted 2C216 from a medical device's network transceiver to an IoMT server, to an authentication service, and finally to a stream service. Data may be further stored in parallel in a database for historical review of the record.

A receiving device client connection 2C217 may authenticate with an authentication service, which may be part of the IoMT Service 300, across a network connection 200 secured by an SSL/TLS certificate 305 and receiving a device tokenized authentication key to establish a secure connection of the transmitting biomedical device to the receiving device. The receiving device utilizes client authorization services to authenticate and authorize the end user for the purpose of accessing patient data and messages from the broadcasting biomedical device.

The receiving device client may receive biological monitoring data through the client data retrieval stage 2C218 by either connecting to the data transmission in real-time or by retrieving historical data by requesting data that is permissioned to the client for retrieval. In a real-time data streaming connection scenario, the stream service may be alerted of a data connection and subsequent client data retrieval 2C218 through a WebSocket or WebTransport QUIC web client protocol. The client connection for receiving live data from the biomedical monitoring device may authenticate by receiving a device granted TLS certificate exchange as described earlier in the IoMT service 300 connection of the remote biological health monitoring system and telehealth real-time live stream system 2A of FIG. 2A. In another scenario, the end user may want to view historical data that they are authenticated and authorized to receive through a client data request.

The medical device may also send a real-time live-stream peer-to-peer biological video and biological audio data to a receiving device 2C219 that may be a digital stethoscope and a digital otoscope as described in the remote biological health monitoring system and telehealth real-time live stream monitoring system 2A of FIG. 2A. The biological video and biological audio streams may also be sent to a video-on-demand (VOD) service for recording. The medical device communicates with an authentication service to authenticate the biomedical monitoring device. The biological audio and video data is then streamed to the receiving device while being recorded and stored in parallel. The receiving device may then communicate with the authentication service to authenticate the end user and subsequently send messages to the medical device for audio and video data streams.

FIG. 2D shows an example sequence diagram for establishing peer-to-peer connection between a transmitting device and a receiving device of a telehealth system. The medical device and receiving device 2D220 authenticate and authorize users to the WebRTC authentication services that receive device and client authentication tokens that may relay tokens to STUN/TURN and Interactive Connectivity Establishment (ICE) services so that the broadcasting medical device client can establish a peer-to-peer connection to the receiving device client. This peer-to-peer connection may be established by the biomedical monitoring device connecting to an authentication service that returns a mutual transport layer security protocol (mTLS) certificate exchange and an authorization token allowing the biological medical streaming device to authenticate against the STUN/TURN and Interactive Connectivity Establishment (ICE) services for the purposes of establishing a real-time peer-to-peer connection between a medical device which may be streaming video, audio and or data to an internet connected client device which may be a web browser or mobile application.

When establishing a video, audio and or data peer-to-peer connection 2D221 from a medical device that can directly stream video, audio, and/or data to a receiving client device, the medical device, which may be a WebRTC-HTTP ingestion protocol (WHIP) creates a session description protocol (SDP) which sends information related to the media being sent. The Interactive Connectivity Establishment (ICE) framework, which describes the network connection information exchange, looks for the lowest latency peer-to-peer connection internet protocol (IP) pathways for the purpose of connecting a receiving device which may view and/or analyze video, listen to and/or analyze audio or receive data from the medical device.

The receiving device, which may utilize a WebRTC-HTTP egress protocol (WHEP) client, or some form an RTCPeerConnection client that has received an SDP answer, which may be in the form of a text string, allowing for a peer-to-peer connection from the receiving device to a broadcasting medical device.

The medical device peer-to-peer video and/or audio data encoded stream transmission 2D222 may communicate biological sensor video or biological sensor audio and data streams directly to the receiving device. Media may also be recorded and played back 2D223 in parallel. The media encoder creates a separate stream ingestion protocol to record audio, video, and data. The medical device data transmissions 2D224 may, in parallel, encode media for sending ingestion streams to cloud storage. The receiving device may also store video on demand.

FIG. 2E shows an example sequence diagram for establishing peer-to-peer connection between a transmitting medical device and a receiving device of a telehealth system using a broadcasting WebRTC-HTTP ingestion protocol (WHIP) client and a receiving WebRTC-HTTP egress protocol (WHEP) client viewer. The session description protocol (SDP) offer exchange 2E225 describes network, media options, ports, transfer protocol and security options. The medical device connects and broadcasts to a WHIP endpoint and CORS URL ingest server, which requests a full list of Interactive Connectivity Establishment (ICE) candidates from the media server broadcasting ingestion URL, which may be described as a streaming service URL. The ICE response must be completed before the SDP answer can respond to the client with an SDP answer for the receiving client.

ICE candidates 2E226, as described in the second stage of the transmitting medical device and a receiving device of a remote biological health monitoring system and/or telehealth system, which establish IP addresses in which the client can connect, whether they are in public STUN, direct IP, or private TURN servers, make a determination request to the media server broadcasting ingestion URL which may be described as a streaming URL, and which sends a response back to the medical device, as well as UDP candidates to the STUN medial server or a private TURN server. In the 3rd stage, the ICE/Datagram Transport Layer Security (DTLS) session connection 2E227 provides unidirectional media flow from the WHIP client to the broadcasting media server. This functionality is based on a current form of media internet broadcasting technology known as Real-Time Transport Protocol (RTP) or Real-Time Messaging Protocol (RTMP) or Real-Time Streaming Protocol (RTSP) which is based on a broadcasting device broadcasting media which may be sent to one or more receiving/viewing capable devices and has a latency of 2-30 seconds. WebRTC technology may provide sub-second latency and is preferred in real-time streaming applications. In the 4th stage, the client request layer 2E228 communicates operations from the medical device's WHIP client, which may be referred to as a media producer or broadcaster, to terminate a session or update ICE operations. In the last stage, the medical device media producer that has sent a one-time SDP request and has received an SDP answer can now establish an ICE/DTLS session with the broadcasting ingestion endpoint of a media server, which may be described as a streaming service URL. Finally, the WHEP media viewer that has received the WHIP ingestion protocol instruction can establish a connection to the producing media server and receive media and/or data unidirectionally.

FIG. 2F discloses a block diagram detailing the internal architecture and capabilities of an all-in-one remote health monitoring and diagnostic medical device. While not explicitly illustrating the physical components, the architecture underscores the device's potential to function as both a digital stethoscope and a digital otoscope, utilizing integrated cameras and audio/microphone sensors. The device may acquire medical audio or video data, displaying it on an integrated touchscreen user interface. Additionally, through a display port-type interface, the device can transmit biomedically related audio, video, and/or images to an extended local user interface receiving device 961, which may be a computer monitor, television screen, or laptop screen. Simultaneously, the device has the capability to transmit this medical audio and video data in real-time to designated remote end-users and their corresponding receiving devices 960 through a secure network connection interface 920 which uses a network transceiver 921 to connect and broadcast the data transmission through an online internet connection.

In parallel, the device analyzes the captured medical video and audio directly on the device itself using artificial intelligence, often referred to as Edge AI or Endpoint AI. Furthermore, the medical device may transmit the camera and audio data to a cloud-based deep learning Large Medical Data Model (LMDM) AI/ML system for a more advanced analysis, which may offer additional insights than what is capable by the onboard edge AI medical device's analysis.

A camera sensor 937A and 937B interfaces with the Camera Serial Interface (CSI) 939A and 939B. The CSI, in conjunction with the Video Input (VI) block, manages the video data, accommodating formats such as YCbCr, RGB, or raw Bayer format data 940. Separately, a microphone sensor 938 connects to the I2S (Inter-IC Sound) 939C interface, facilitating the capture and transmission of audio data. This data undergoes its own processing pathway, distinct from the video data. The raw Bayer format video data from the VI or CSI block is processed by the Image Signal Processor (ISP) 941, converting it into YUV output 942. Advanced image processing techniques are employed to refine the input and mitigate artifacts potentially introduced by a camera sensor.

The processed video and audio data are then stored and managed within the memory controller fabric 922B. This fabric serves as a central management system for data, ensuring efficient storage and accessibility for further processing. It interacts with the Sensor IO 936, a higher-level hardware interface that acts as a gateway, facilitating communication between various sensors and the device's processing components. Once the video/image data is processed and stored, it is routed to the compositor VIC (Video Image Compositor) 943. The compositor VIC is equipped to handle various system UI scaling, blending, and rotation operations, among other advanced functions. Audio signals from the I2S interface are refined and optimized by the Audio Processing Engine (APE) 965, a self-contained unit designed for Ultra Low Power (ULP) audio processing.

Following these processing stages, the refined data is represented as Audio/Video RGBA 944 within the controller fabric. This processed data then has a direct connection to the Multimedia Complex 930, which efficiently manages audio and video data, further enhanced by a hardware accelerator 931.

The Multimedia Complex 930 encompasses several integral components for multimedia processing. The Video/Audio Decoder 933 decodes incoming multimedia streams, preparing them for subsequent operations. The Video/Audio Encoder 935 compresses processed multimedia data for storage or transmission. The HDR processor 932 offers dynamic range adjustments for visual data. Notably, the Hardware Accelerator 931, leveraging multi-core GPU and multi-core CPU, significantly enhances the efficiency of the encoding and decoding processes. In this way, hardware acceleration is pivotal in delivering real-time performance, especially in applications demanding high-speed multimedia processing which may be video and/or audio coming from the remote health monitoring device.

Following the processing within the Multimedia Complex 930, the encoded multimedia data from the Video/Audio Encoder 935 is directed to the Video/Audio Sinks 945. These sinks serve as intermediaries, channeling the processed data to the appropriate display or output mechanisms. The onboard display 952 may display the video/audio sink integrated within the medical device, acts as the primary interface for immediate visualization and auditory playback of the processed medical data. This ensures that users have an immediate and precise representation of the acquired biomedical information.

The processed data also has the potential to be routed externally. The DisplayPort (DP) 947 is available for external monitor connections 961, providing a broader visualization platform for detailed medical analysis. The Embedded DisplayPort (eDP) 948, on the other hand, is dedicated to the device's onboard display, ensuring a seamless and high-quality display experience for the user.

From the Video/Audio Sink 945, the encoded audio/video data from the encoder 935 is channeled to a streaming source encoding processor pad 953. This pad may initiate a multi-threaded stream source 954. Subsequently, the pad source interfaces with a tee 955 which is a specialized encoding element that takes the initial thread and divides it into multiple threads. These threads facilitate the processing of a file sink in diverse ways, enabling simultaneous streaming sink and processed file outputs. The subsequent pad utilizes the sources from the previous step, specifically 957E, 958E, and 959E, as the sinks 957F, 958F, and 959F. The sinks are then used for the pad settings 957ST, 958ST, and 959ST, which dictate the specific processing and conversion parameters for the processing sink media data. The sources from the previous pad namely, 957G, 958G, and 959G, are channeled to these final sinks 957H, 958H, and 959H. The ultimate representation of the processed data is categorized into three primary outputs: Stream 957S, Local 958S, and Display 959S. The Stream 957S is tailored for real-time streaming connections that may use peer-to-peer broadcasting technology to transmit data to a receiving device described in FIG. 2B, FIG. 2C, FIG. 2D, and FIG. 2E. The Local source 958S retains a copy of the video and/or audio file which may be used to transmit the data to a data retention service for on-demand availability. The display source 959S is dedicated to rendering the data on the onboard display 952 via the eDP device hardware connection 948.

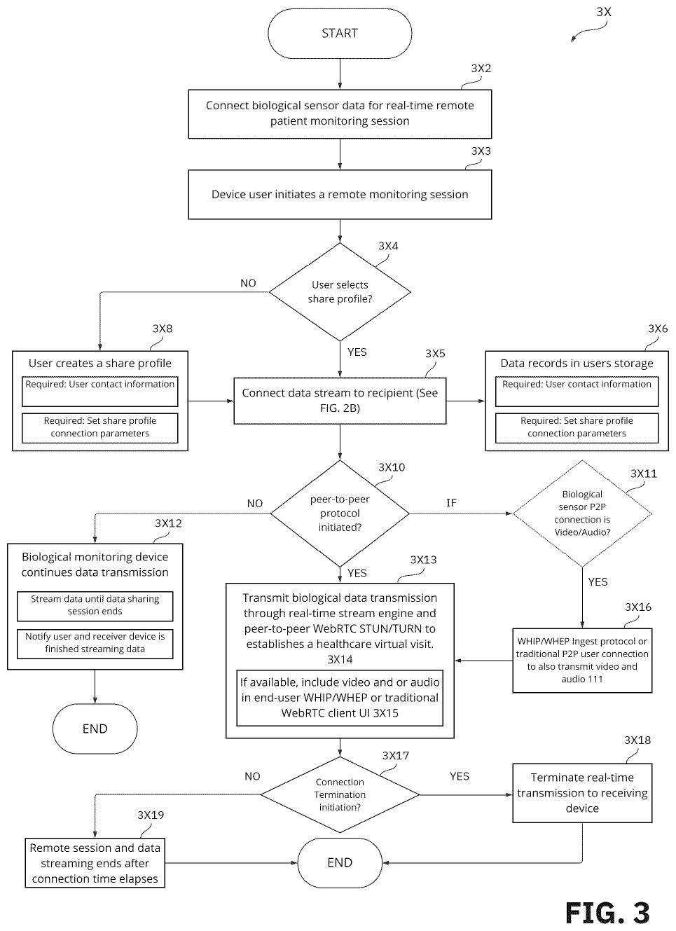

FIG. 3 is a high-level flowchart illustrating an example method 3X for live streaming data via a central server or peer-to-peer technology. Biological sensor data is connected for a real-time remote patient monitoring session 3X2. The device user initiates a remote monitoring session 3X3 and either selects a share profile 3X4 or creates a share profile 3X8 for the purpose of allowing a permissioned caregiver or medical staff member to remotely control and/or observe the patient biological-monitoring data. When creating a share profile, the user contact information and share profile connection parameters are required. Once a share profile is selected, a data stream is connected to a recipient 3X5, which either records data in the user's storage 3X6 or initiates a peer-to-peer health monitoring medical device data connection 3X10, which may broadcast a patients biological sensor data 3X13. If a peer-to-peer protocol is not initiated, the biological monitoring device may continue data transmission 3X12, which streams data until the data sharing session ends and notifies the user and receiver that the device has finished streaming data. The data may be available to the authorized recipient for the purpose of reviewing the data at another time. Any user described time limits, and other shared data attributes, will follow the instruction for viewing a patient's historical data on-demand. If a peer-to-peer protocol has been initiated, biological sensor data is transmitted through a real-time stream engine and a peer-to-peer real-time telehealth visit may be established in a separate peer-to-peer connection 3X14, which may also include video and/or audio in an end-user WHIP/WHEP user interface or traditional WebRTC user interface 3X15. If connection termination is initiated 3X17, real-time transmission to the receiving device is terminated 3X18, otherwise the remote session and data streaming end after the connection time elapses 3X19.

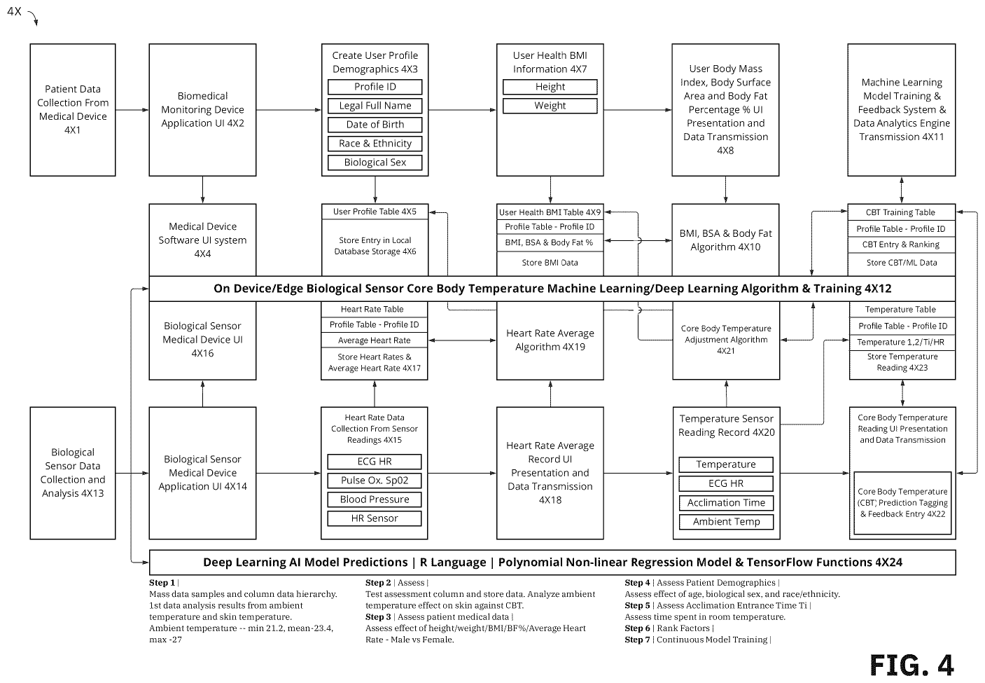

FIG. 4 is a high-level flowchart illustrating an example method 4X for using artificial intelligence (AI) algorithms and a Machine Learning (ML) Large Medical Data Model in coordination with a medical device's biological sensor data to increase sensor accuracy, which may consist of raw sensor data or raw sensor data that has been adjusted for sensor predictions. An example of this is body temperature from the skin of the forehead and adjusting it to the core body temperature 4X21, which might otherwise be accomplished by using linear algorithmic functions and may give inaccurate results. The illustrated process involves taking individual patient medical device readings and integrating them with the patient's own comprehensive medical data. The combined data is then processed through AI/ML algorithms to correct and/or predict accurate sensor readings 4X21. Patient data may be collected from the medical device itself 4X1 through an on-device application and user interface 4X2. A user profile may be created on the device and the user may include their demographic information 4X3 and health information 4X7, which may include the user's age, date of birth, height, and weight; this information may be acquired from an external data transmission relating to the user's health data. Data related to the user's body mass index, body surface area, and body fat percentage are gathered 4X9 and sent to the health profile storage on the medical device. The data is subsequently processed by the device's AI/ML 4X12 algorithms, which may be referred to as Edge AI or Endpoint AI, for immediate analysis or inference. The data analysis on the device may provide a feedback mechanism 4X11 of the artificial intelligence, which may come from direct data analysis or from an inferenced decision-making result. The medical device tracks both the resulting artificial intelligence response and any feedback acquired and subsequently transmits the data to a machine learning and artificial intelligence layer, which may be a server system consisting of compute and machine learning/artificial intelligence processing units. To keep the on-device artificial intelligence current, the large medical data model can be fine-tuned, trained, and released as an update 4X12 back to the medical device, providing increased accuracy and better inference over time. Deep learning artificial intelligence model predictions 4X24 can be generated by (1) obtaining mass data samples and column data hierarchy from sensors; (2) assessment and analysis of ambient temperature effect on skin against CBT; (3) assessment of patient medical data and the effect of height, weight, BMI, body fat percentage, and average heartrate for male and female patients; (4) assessment of patient demographic information; (5) assessment of acclimation entrance time; (6) ranking of factors; and (7) continuous model training.

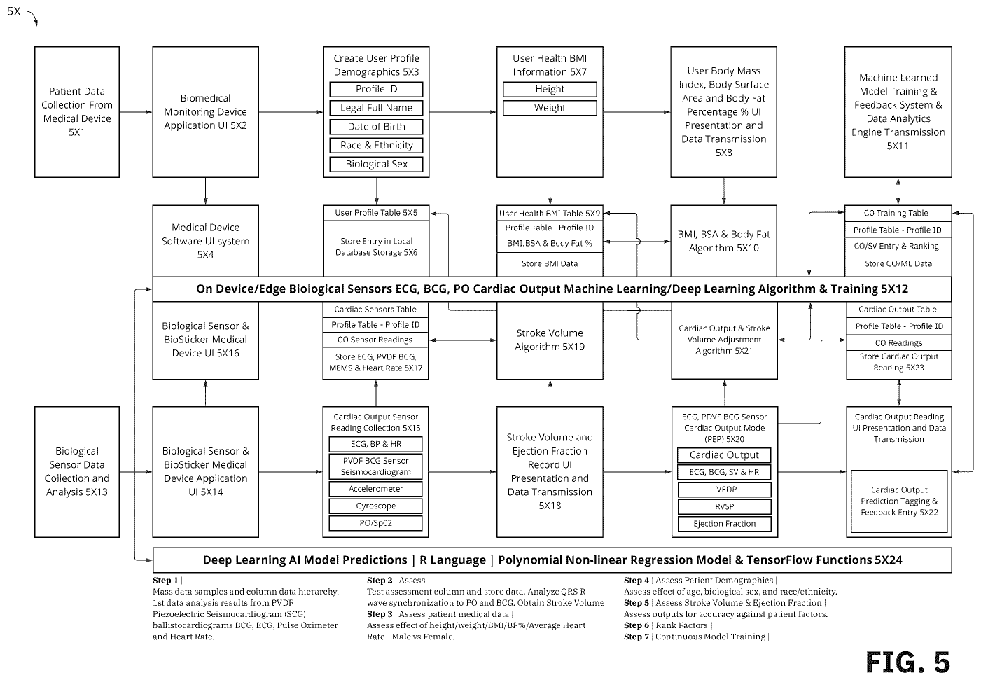

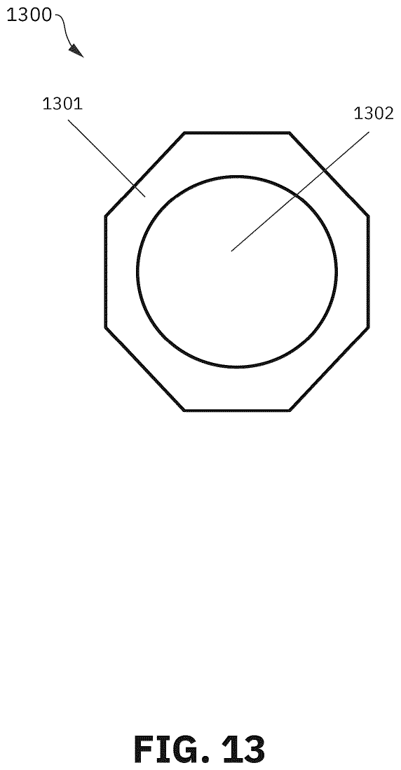

FIG. 5 is a high-level flowchart illustrating another example method 5X for using artificial intelligence (AI) algorithms and a Machine Learning (ML) Large Medical Data Model in coordination with a medical device's biological sensor data to increase sensor accuracy, which may consist of raw sensor data or raw sensor data that has been adjusted for cardiac output predictions. The medical device, which may include biological sensors consisting of heart rate sensors, an ECG sensor, and a pulse oximeter (PO) sensor, which may communicate with an additional adapter medical device 5X14 (See FIG. 13), which may include biological sensors, consisting of ballistocardiogram (BCG) sensor, polyvinylidene difluoride (PVDF) piezoelectric sensors, piezoelectric seismocardiogram (SCG) sensor, an ECG sensor, and heart rate sensors 5X15. This may be used for the purpose of obtaining the patient's cardiac output, including stroke volume and heart rate 5X17. When obtaining a patient's stroke volume 5X18 non-invasively, artificial intelligence 5X19 analyzes readings from an SCG and/or BCG, which correlates the QRS complex ECG intervals to the detected information found through sensor readings, creating an analysis of the pre-ejection period (PEP) 5X20 consisting of the elapsed time between the depolarization of the left ventricle, which viewed as an ECG QRS wave and the start of the ventricle ejection of blood flow. This represents the period of left ventricle contraction and cardiac valve closing in which the BCG obtained heart sounds that overlay with the corresponding ECG to predict cardiac output.

The illustrated process involves taking individual patient medical device readings and integrating them with the patient's own comprehensive medical data. The combined data is then processed through AI/ML algorithms to correct and/or predict accurate sensor readings 5X21. Patient data may be collected from the medical device itself 5X1 through an on-device application and user interface 5X2. A user profile is created on the device and the user may include their demographic information 5X3 and health information 5X7, which may include the user's age, date of birth, height, and weight; this information may be acquired from an external data transmission relating to the user's health data. The user's body mass index, body surface area, and body fat percentage data are collected and transmitted to the health profile storage on the medical device 5X9. The data is subsequently processed by the device's AI/ML 5X12 algorithms, which may be referred to as Edge AI or Endpoint AI, for immediate analysis or inference. The data analysis on the device may provide a feedback mechanism 5X11 of the artificial intelligence, which may come from direct data analysis or from an inferenced decision-making result. The medical device tracks both the resulting artificial intelligence response and any feedback acquired and subsequently transmits the data to a machine learning and artificial intelligence layer, which may be a server system consisting of compute and machine learning/artificial intelligence processing units. To keep the on-device artificial intelligence current, the large medical data model can be fine-tuned, trained, and released as an update 5X12 back to the medical device, providing increased accuracy and better inference over time.

Deep learning AI model predictions may be generated by 5X24 (1) obtaining mass data samples from a polyvinylidene difluoride (PVDF) piezoelectric sensor, BCG, SCG, ECG, pulse oximeter and heart rate sensors 5X15; (2) assessment and analysis of QRS R wave synchronization to PO and BCG; (3) assessment of patient medical data and the effect of height, weight, BMI, body fat percentage, and average heartrate for male and female patients; (4) assessment of patient demographic information which assesses the effect of age, biological sex, race, and ethnicity; (5) assessment of stroke volume and ejection fraction, which assesses outputs for accuracy against patient factors; (6) ranking of factors; and (7) continuous model training.

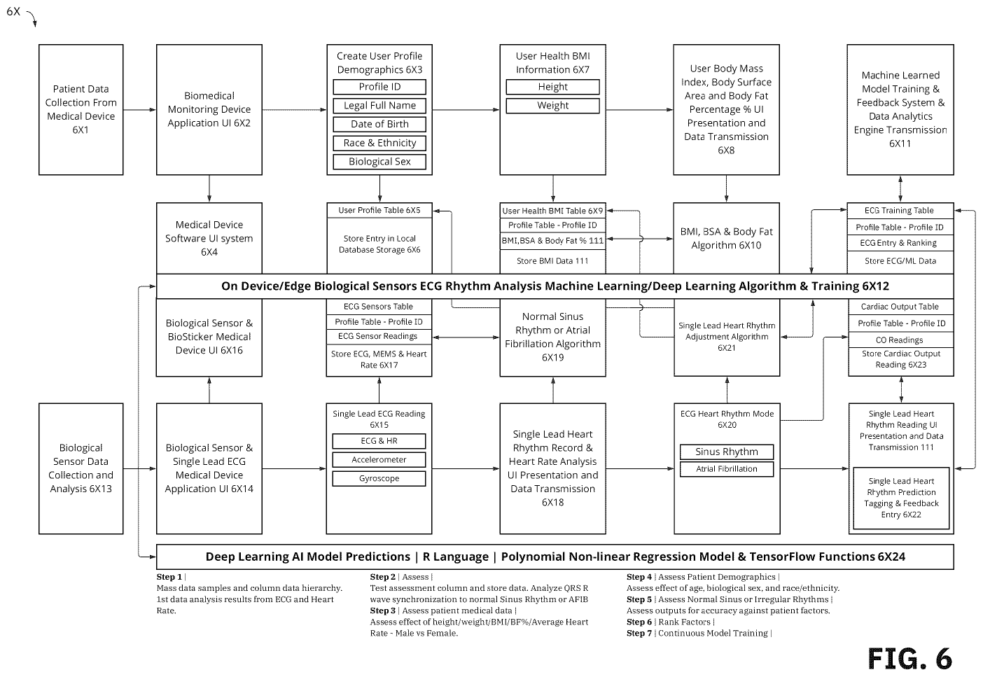

FIG. 6 is a high-level flowchart illustrating another example method 6X for using artificial intelligence (AI) algorithms and a Machine Learning (ML) Large Medical Data Model in coordination with a medical device's biological sensor data 6X13 to increase sensor accuracy, which may consist of raw sensor data or raw sensor data that has been adjusted for sensor predictions. Aa illustrated, a medical device with a single channel ECG sensor attains an ECG reading 6X15 of a patient and can detect a normal sinus rhythm or an irregularly irregular arrhythmia 6X19. As described in FIG. 4 and FIG. 5, both patient health information and medical device readings may be applied to an artificial intelligence algorithm to increase the diagnostic prediction of an ECG reading 6X21.

Deep learning artificial intelligence model predictions 6X24 can be generated by (1) obtaining mass data samples and column data hierarchy from sensors; (2) assessment and analysis of ambient temperature effect on skin against CBT; (3) assessment of patient medical data and the effect of height, weight, BMI, body fat percentage, and average heartrate for male and female patients; (4) assessment of patient demographic information which assesses the effect of age, biological sex, race, and ethnicity; (5) assessment of acclimation entrance time; (6) ranking of factors; and (7) continuous model training.

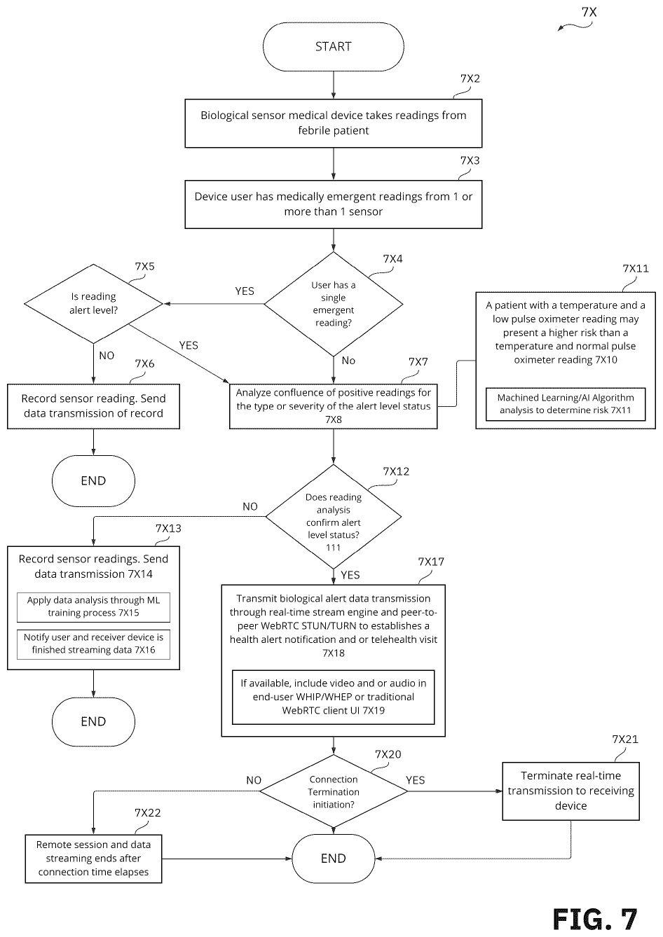

FIG. 7 is a high-level flowchart illustrating an example method 7X for analyzing data from a health monitoring device and a patient's health characteristics to determine a possible emergency alert notification. In this example, a medical device takes biological sensor readings from a patient that may be febrile 7X2. The device may obtain a medically emergent reading from at least one sensor 7X3. Where a user has a single medically emergent reading 7X4, the reading is analyzed to determine whether it has reached an alter level status 7X5. If the reading has not reached the status of an alert level, then a recorded sensor reading is recorded without the status as an alert 7X6. When there is a reading that reaches an alert level 7X7, it is analyzed against other readings to determine the severity of the alert level based on multiple readings 7X8. For example, where a patient has a high temperature 7X10, but a normal pulse and normal heart rhythm, the alert level status may not require a response. In another example, where a patient has a high temperature, high pulse rate, and an erratic hearth rhythm, the alert status may require an emergency response. This is determined by machine learning and artificial intelligence algorithms directly on the device 7X11, which analyses the confluence of positive medical device readings for the type of severity of an alter level status 7X7. If the alert level status is not confirmed 7X13, the sensor readings are recorded 7X14 and transmitted for machine learning data analysis and artificial intelligence model training 7X15. A notification 7X16 is sent to the user's medical device and the receiving device to notify the user and receiver that the device has completed its data transmission.

If, however, the alert level is confirmed 7X17, biological alert data is transmitted through a real-time stream engine and a real-time peer-to-peer data transmission establishes a health alert notification and/or a telehealth visit 7X18. If available, audio and/or video data may be included and transmitted to the end-user 7X19. Subsequently, a connection termination signal may be initiated 7X20 to terminate the remote session 7X21, which may be initiated by the operator of the medical device, the end user on the receiving device, or the time limit 7X22 established for the connection.

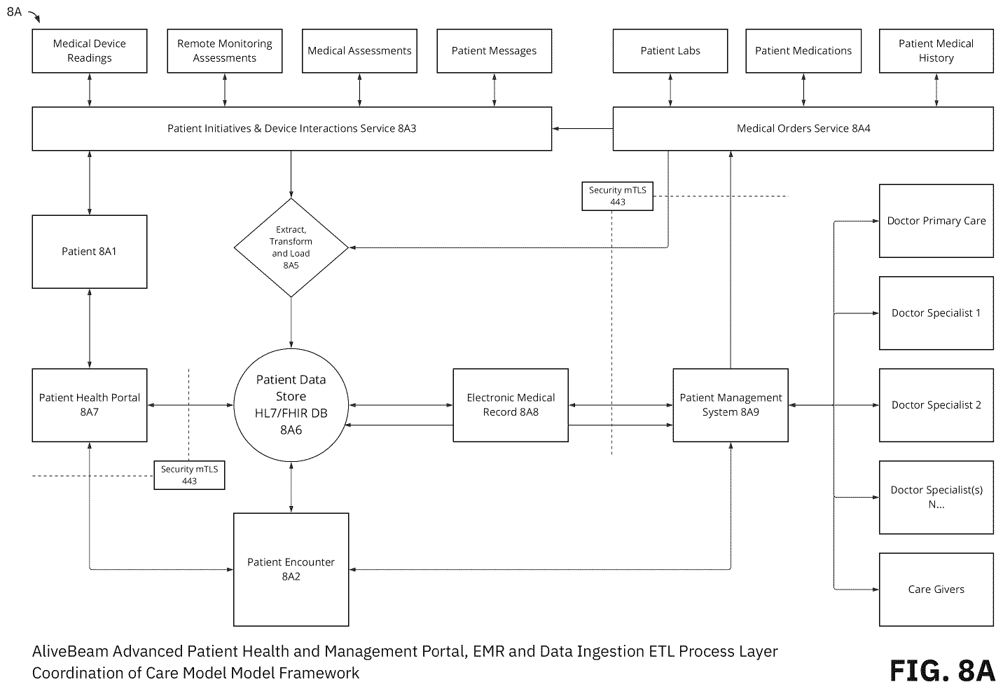

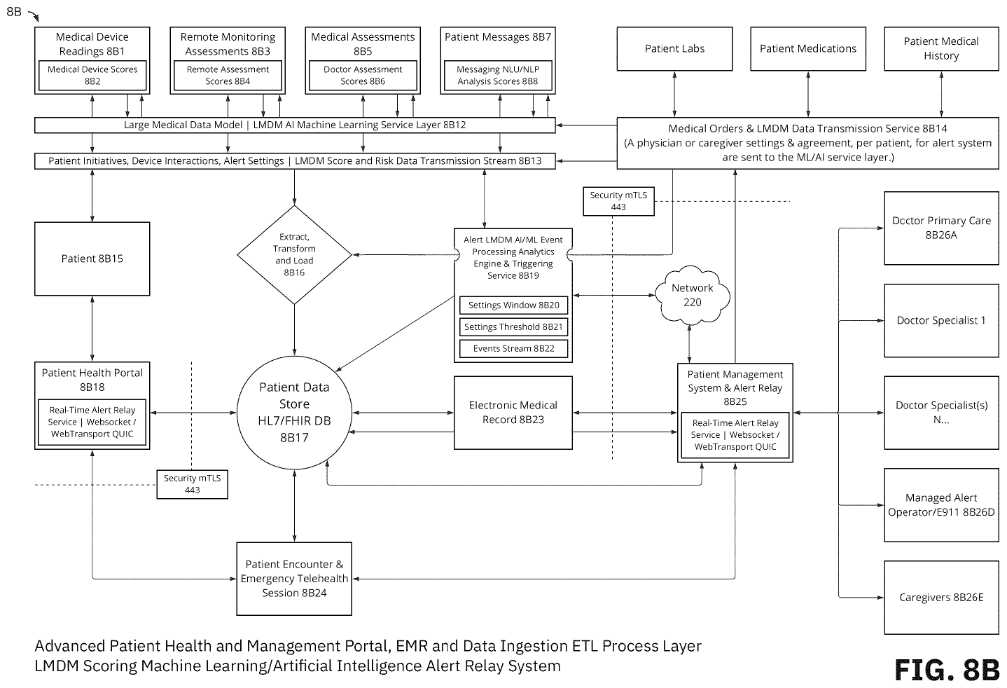

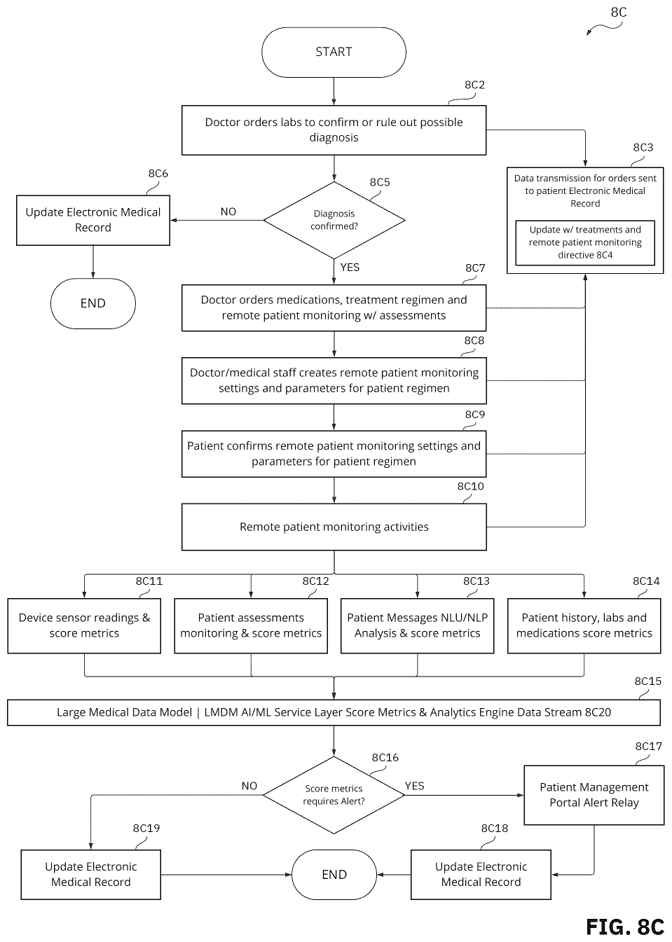

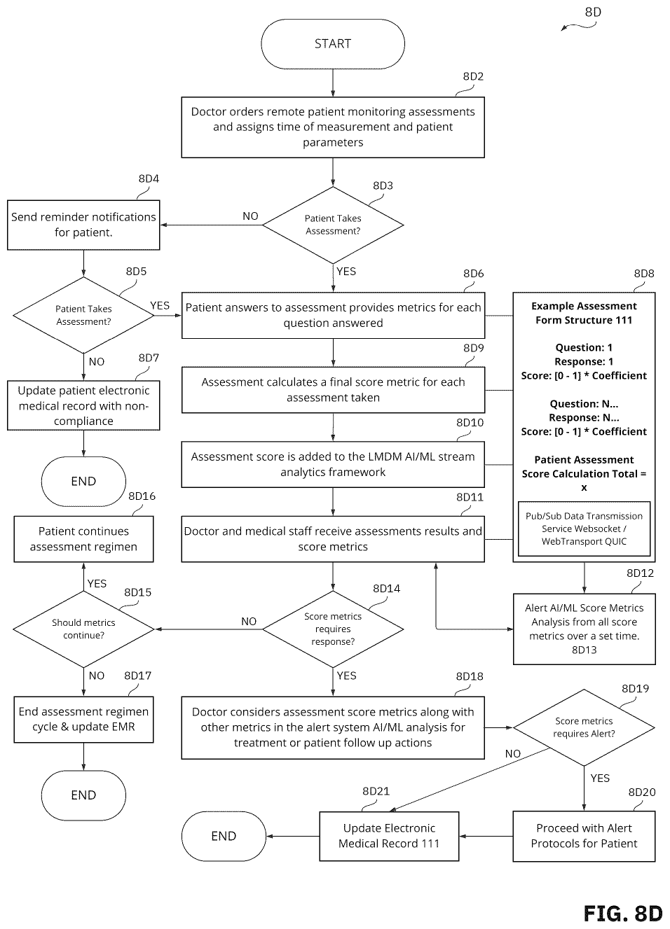

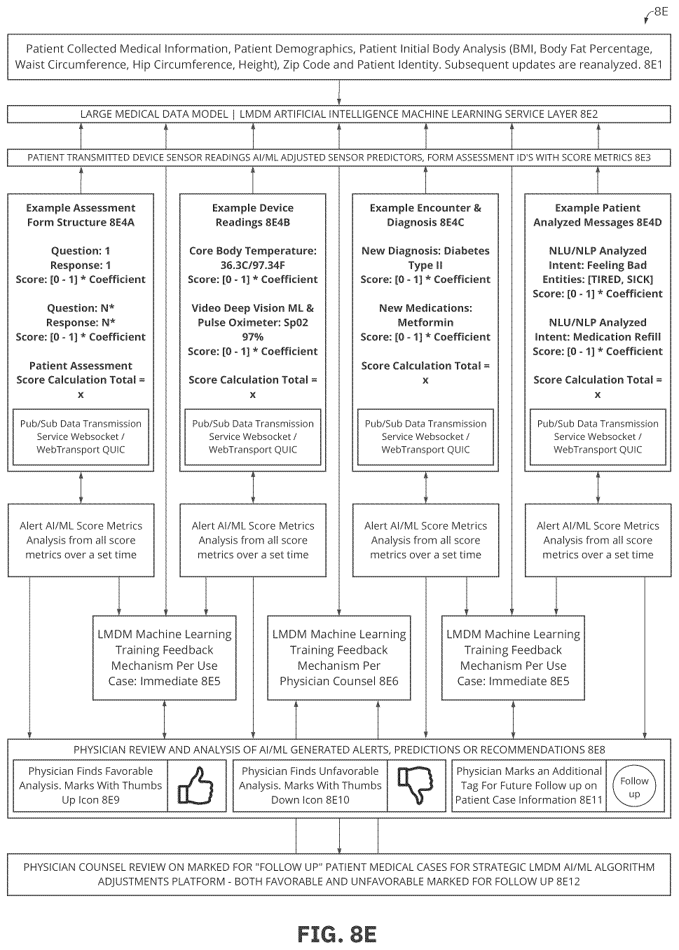

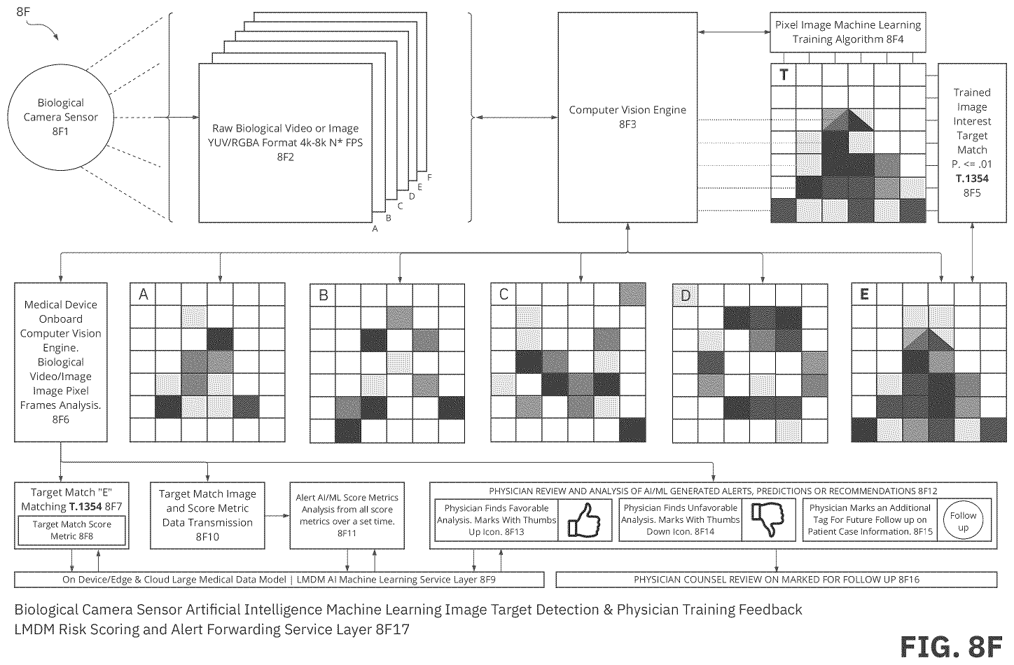

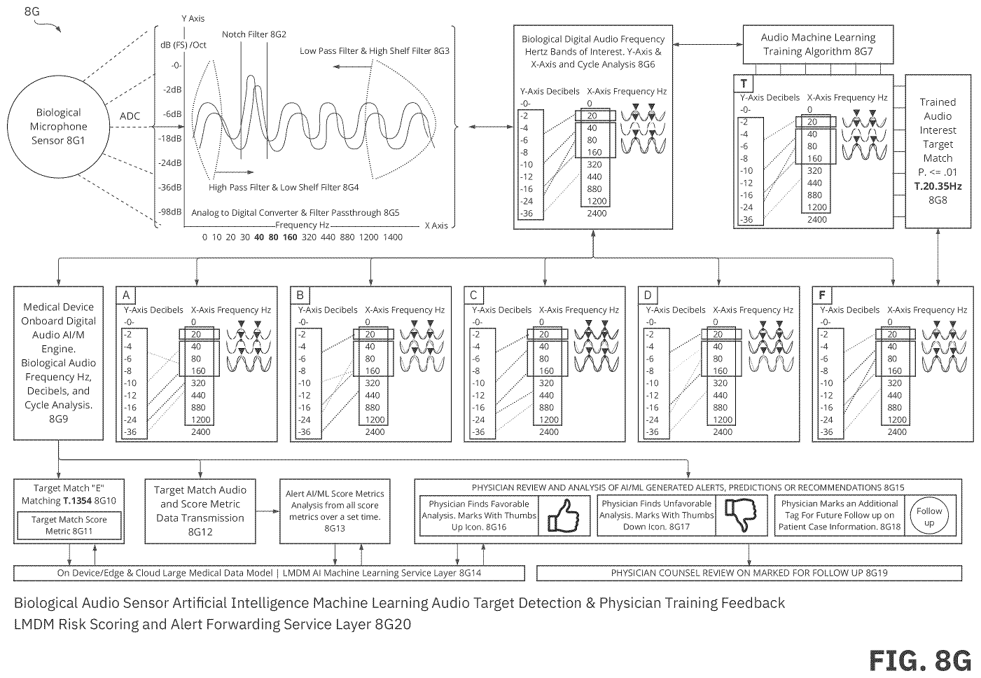

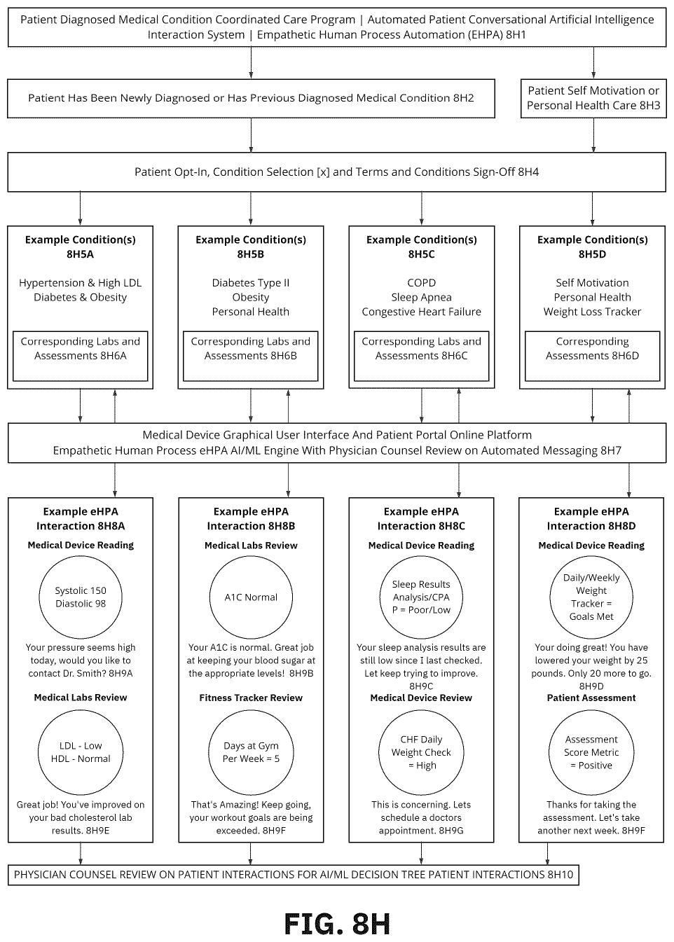

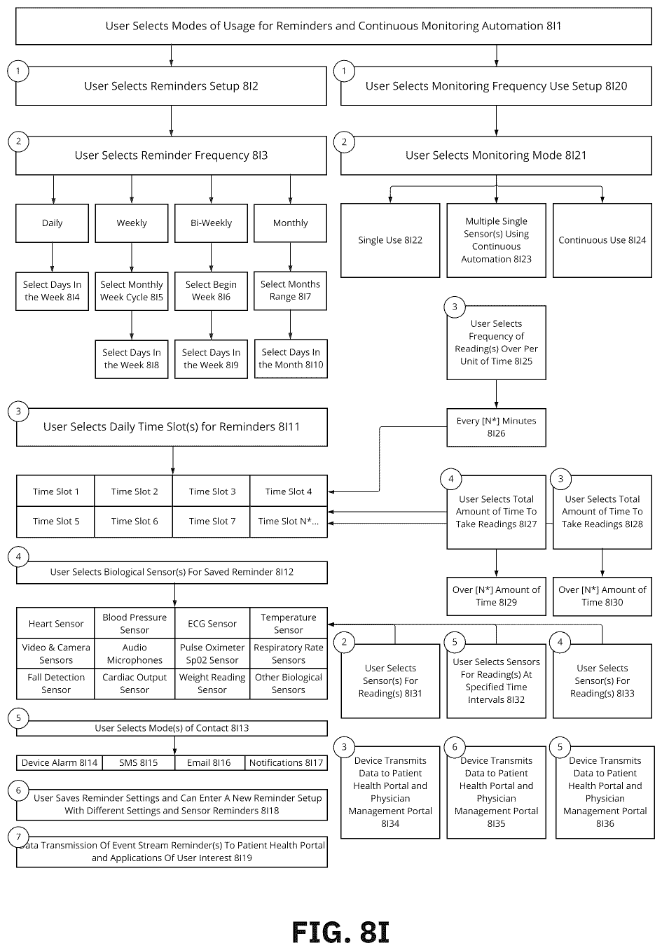

FIG. 8A is a schematic representation of the patient data management portal, electronic medical record (EMR), a data ingestion extract, transform, and load (ETL) process, and coordinated care model framework 8A. This is where a patient 8A1 is assigned patient health tasks and initiatives, which may come from a doctor's orders and/or a patient medical care-plan 8A4, that include an array of medical observations and assessments.

Patient data 8A6, which may include remote patient monitoring medical device readings of a health monitoring device, cognitive automation monitoring assessments, medical assessments, and/or patient messages are obtained on an ongoing basis; additionally, information from one or more medical encounters 8A2 such as patient laboratory results, patient medications, and past medical history are collected in a patient initiatives and medical device interactions service 8A3 that further processes the data into an ETL process 8A5 which then loads the data into the patient data store 8A6 and EMR 8A7.

The medical data collected is owned and controlled by the patient and authorized for usage by one or more medical providers. This includes any acting patient health portal 8A7, EMR 8A8, and/or patient management portal 8A9. Medical data and health information has the potential to be shared among all connected health care providers through patient authorization.

In this embodiment, a patient might have an encounter 8A2 with a medical professional, potentially leading to a diagnosis and medication order 8A4. This professional could be a general practitioner. Subsequently, the same patient might have another interaction with a physician specialist from a different health system. As the patient data is collected into the patient data store, the data is in turn authorized for use by the patient in the patient health portal 8A7 and by medical professionals in an electronic medical record (EMR) portal 8A8 and patient management system (PMS) portal 8A9.

FIG. 8B is a schematic representation of the patient data management portal, electronic medical record (EMR) and a data ingestion extract, transform, and load (ETL) process, and coordinated care model framework SB. This is where a patient is assigned patient health tasks, which may come from a doctor's orders 8B14 and/or a patient medical care plan, that may include an array of medical observations and assessments. Furthermore, a Large Medical Data Model machine learning service layer 8B12 analyzes patient data for the purpose of providing medical alerts and patient health insights from a confluence of data points.

Patient data 8B17, which may include remote patient monitoring medical device readings of a remote health monitoring device 8B1 and its subsequent scoring metrics 8B2, cognitive automation monitoring assessments 8B3 and their subsequent scoring metrics 8B4, medical assessments 8B5 and their subsequent scoring metrics 8B6, and/or patient messages 8B7 and their subsequent scoring metrics 8B8 are obtained on an ongoing basis. Additionally, information from one or more medical encounters such as patient laboratory results, patient medications, and past medical history are collected in a patient initiatives and medical device interactions service 8B24 that further processes the data into an ETL process 8B16 which then loads the data into the patient data store 8B17 and EMR 8B23.

The medical data collected is owned and controlled by the patient 8B17 and authorized for usage by one or more medical providers. This includes any patient health portal 8B18, Electronic Medical Record (EMR) 8B23, and/or patient management portal 8B25. Medical data and health information has the potential to be shared among all connected health care providers through patient authorization.

In this embodiment of the present invention, a patient may have an Encounter 8B24, which may include a subsequent diagnosis and medication order, with a medical professional who may be a general practitioner and a patient may have another encounter with medical professional who may be a physician specialist from another health system. As the patient data is collected into the patient data store 8B17, the data is in turn authorized for use by the patient in the patient health portal 8B18 and by medical professionals in an electronic medical record (EMR) 8B23 portal and patient management system (PMS) portal 8B25.

Additionally, patient data that has been identified to be of an emergent alert level status from the artificial intelligence algorithm can trigger 8B19 an alert relay messaging system, which the alerting system may relay the alert to and from the monitoring system in the Patient Management System (PMS) 8B25, using peer-to-peer technologies that can transmit messages in real-time, such as WebRTC and/or WebTransport QUIC or Websocket, and may be sent to a medical professional 8B26E and/or caregiver 8B26E that has been authorized to receive alert notifications from a confluence and analysis of health data. The patient alert may be relayed to a managed alert operator system such as an Enhanced 911 service 8B26D.

FIG. 8C depicts a flow diagram outlining a method implemented by a system 8C. The method commences with a patient's consultation with a medical doctor to confirm or dismiss a potential diagnosis 8C2. Upon confirmation of a diagnosis 8C5, the physician may prescribe medications 8C7, initiate a treatment regimen, which may include medical assessments, and/or activate remote patient monitoring. Subsequently, medical professionals set the remote patient monitoring parameters 8C8 for the patient's health monitoring device and provide instructions associated with the patient's regimen.

The patient validates these settings and parameters 8C9 either on the health monitoring device or via an associated online mobile or web platform. Following this, the patient engages in the prescribed remote monitoring activities 8C10. Simultaneously, the Electronic Medical Record (EMR) updates 8C3 to reflect the treatment care-plan and the remote patient monitoring directives 8C4.

Each engagement with the medical device generates biological sensor readings and corresponding scoring metrics 8C11. Additional inputs, including patient assessments 8C12, patient messages 8C13 which may be analyzed using Natural Language Understanding (NLU), Natural Language Processing (NLP), and/or Generative Artificial Intelligence (GAI/LLM), patient medical history data, laboratory results, and medication details 8C14, are integrated into scoring metrics at each data point and during each question/response interaction. Subsequently, the data is analyzed by a large medical data model (LMDM) 8C15 alongside other artificial intelligence and machine learning algorithms 8C20 to determine the potential of alert-level medical events 8C16.

If no alert is deemed necessary 8C19, the EMR updates with the current patient data score, concluding the interaction. If an alert-level medical event is detected 8C17, a notification is sent to the patient management system portal. The method concludes with the transmission and update of the patient's data in their EMR 8C18.

FIG. 8D depicts a flow diagram illustrating a method implemented by a system 8D. The method begins with a physician's medical order 8D2 for remote patient monitoring assessments. These assessments 8D8 might be custom designed by the physician or sourced from a repository of patient monitoring care. Such assessments can span various medical domains, from behavioral or mental health to general medical care, of which may include Social Determinates of Health (SDoH). Other assessments may include standardized medical assessments recognized within the medical community such as the Patient Health Questionnaire-9 (PHQ9) which serves as a depression screener for patients.

Subsequently, the patient takes an assessment 8D3. In instances where the patient may not comply a reminder notification 8D4 may be sent to the patient to encourage adherence. Continued non-compliance 8D7 results in the patient's medical record being updated with a non-compliance assessment record which may initiate a follow-up action by the medical care team.

When the patient completes the assessment, each question, and its corresponding response yield scoring metrics 8D6. These metrics collectively determine the final score of the assessment 8D9. The assessment scores are relayed to an artificial intelligence large medical data model (LMDM) algorithm for data analysis 8D10, providing insights into the patient's medical information.

Pertinent information, which may encompass assessment outcomes and associated scoring metrics, is dispatched either instantaneously or over a designated remote patient monitoring interval to the receiving medical staff and/or physician 8D11.

In situations where the patient's scoring metrics do not indicate an imminent medical concern 8D14 but still necessitate continued monitoring 8D15, the patient simply persists with the assessment and remote monitoring routine 8D16. If the metrics analysis by artificial intelligence suggests that ongoing monitoring is not essential, the assessment cycle concludes and the patient's Electronic Medical Record (EMR) 8D17 is updated.

Should the patient's scoring metrics indicate a potential medical emergency 8D12, a physician evaluates these metrics in conjunction with other system alerts 8D13. The concerning physician or medical staff 8D18 may consider assessments score metrics along with other metrics in the AI/ML analysis to inform treatment decisions, clinical support, and any requisite patient follow-up actions.

If an emergent medical alert status 8D19 is detected from the patient's data analysis, appropriate protocols may be activated 8D20 which may subsequently update the patient's EMR 8D21.

FIG. 8E depicts a flow diagram for patient interactions, which include a medical assessment, medical device readings, medical encounters, patient conditions, diagnosis, medications, laboratory results, generally observed health information from a patient, and/or messaging to a provider 8E1, for the purpose of scoring each data point, that is adjusted by a optimization algorithm, which tries to find the best coefficient for a certain model which can used for analyzing each data point against an artificial intelligence and machine learning medical alert and notification and relay system. For example, a patient health questionnaire-9 (PHQ9) can be used as a depression screening assessment to help diagnose and/or monitor a patient's mental or behavioral state. In this screening assessment example 8E4A, there is a prescribed scoring mechanism for each question and response. This scoring mechanism is designed to be applied to an artificial intelligence and machine learning model 8E2. In this instance, another scoring mechanism is applied for each question and response that goes through an artificial intelligence and machine learning model 8E2. Along with a coefficient adjustment, the scoring mechanism is now able to act as a weight of importance for each data point that is included in the artificial intelligence algorithms for the purpose of redirecting and detecting the heath alert by one or more data point from a patient interaction. The patient interaction may be transmitted to an AI/ML model to adjust corresponding sensor predictions 8E3.

Interactive machine learning (IML), or more specifically human-in-the-loop, provides interactive and constant feedback on health alerts generated by the system 8E5. This feedback is subsequently employed to enhance and further train the medical model 8E3. Other data points from device sensors, patient assessments, patient messaging, and patient history may be collected over time and subsequent scoring metrics may result from each data point collected. Where an alert is required, the patient management portal alert relay updates the electronic medical record. At least one medical professional reviews and analyzes alerts, predictions, and/or recommendations within a feedback review system 8E8. Within this system, human interactions designated as favorable 8E9 or unfavorable 8E10 are logged for further analysis. When specific deterministic combinations of incoming data points are encountered, particularly those confluences that meet defined thresholds or criteria, algorithmic protocols may automatically mark these for follow-up 8E11. Additionally, physicians can provide direct annotations on these data points, further indicating the need for follow-up 8E11. The collected data is then periodically reviewed by a medical data analytics team 8E12, composed of physicians and data scientists, delving into health data points, which may lead to fine tuning of the AL/ML algorithms or adjusting the data collection to remove outlier data providing poor results. Additional analysis may also be used for bias testing, model improvement and correcting bias within the model. Samples with unfavorable designations are given similar attention. After the review process, the insights gathered are used to refine and train the AI/ML system for enhanced accuracy and performance.

FIG. 8F is a schematic depiction of a system comprising a biological video sensor utilizing artificial intelligence and machine learning for video or image that may analyze video or images for possible medical pathology. Biological camera sensors 8F1 may input raw biomedical video or digital image frames 8F2 into a computer vision AI/ML engine 8F3, which conducts a frame-by-frame analysis, which may be described as frame extraction. Once the frame is extracted, computer vision algorithms identify specific features in the image 8F4, which may be described as feature detection. Features can be corners, edges, textures, color, and/or other unique patterns in the image. For example, color may provide the ability to determine medical pathology and give a contrast between normal tissue examples versus abnormal tissue examples that are detected in the image. Once the algorithm has detected a feature in the image 8F5, the algorithm is used to add a descriptor for each feature, which acts as a fingerprint, or unique identifier that distinguishes it from other features, which may be described as descriptor extraction.

Once features and their descriptors are extracted, they are compared to a database of known targets 8F11. The AI/ML algorithm attempts to find the closest matching features between the current frame and the known target features. A target match score is generated and transmitted to a large medical data model (LMDM) AL/ML algorithm for score metrics analysis, which may be used to determine, directly on the device 8F6, if there is a medical concern or an emergent medical condition, which may be described as Edge AI or Endpoint AI. The objective is to compare a target image match 8F7 with the associated target match score 8F8 and determine its medical relevance. The system 8F10 assesses if there's a correlation with known medical conditions from the patient's medical history, leading to potential emergency medical alerts, diagnostic indicators, or general diagnoses 8F9. At least one medical professional reviews and analyzes alerts, predictions, and/or recommendations within a feedback review system 8F12. Within this system, human interactions designated as favorable 8F13 or unfavorable 8F14 are highlighted for further analysis. The collected data is then periodically reviewed by a medical data analytics team 8F15, composed of physicians and data scientists, delving into health data points, which may lead to fine tuning of the AL/ML algorithms or adjusting the data collection to remove outlier data providing poor results. Additional analysis may also be used for bias testing, model improvement and correcting bias within the model. Samples with unfavorable designations are given similar attention. After the review process 8F16, the insights gathered are used to refine and train the AI/ML system for enhanced accuracy and performance 8F17.

FIG. 8G is a schematic depiction of a system comprising a biological audio sensor 8G1 utilizing artificial intelligence and machine learning to analyze sound waves for potential medical pathologies. Acting as an advanced digital stethoscope, the biological audio sensor may capture and input raw biomedical audio data or digital audio samples into an audio analysis AI/ML engine. Sound waves are converted from analog to digital, which may be known as analog to digital conversion (ADC) 8G5. This allows the audio wave form to become a discrete digital representation of the signal.

Initially, the digital signal is processed through a low-pass filter 8G3, which attenuates frequencies above a certain cutoff. This is complemented by a high-shelf filter, which further attenuates frequencies above a specific point, enhancing the overall reduction in high-frequency components. In coordination, a high-pass filter 8G4 attenuates frequencies below a designated threshold, and this can be coupled with a low-shelf filter that further reduces those frequencies. The combined action of these filters ensures a comprehensive shaping of the signal by attenuating both undesired high and low-frequency content. A notch filter 8G2 can also be incorporated for specific frequency bands as needed 8G6. The resultant baseline audio output can then be analyzed further downstream in the pipeline process 8G7.

The signal can be analyzed by applying a Discrete Fourier Transform (DFT) which may utilize the Fast Fourier Transform (FFT) algorithm. A process known as Short-Time Fourier (STFT) or one of its variants, may be used to divide the audio signal into short, overlapping segments, which compute the Fourier Transform for each segment, allowing for the analysis of how the frequency content changes over time.

The engine then conducts a segment-by-segment analysis, described as “audio segmentation.” Once an audio segment is isolated, audio analysis algorithms identify specific characteristics within the sound, termed feature detection. These characteristics encompass aspects like spectral centroid, zero-crossing rate, frequencies, harmonics, and the unique timbre patterns present in the audio signal. For instance, certain spectral properties might be indicative of typical heart or lung sounds, facilitating the differentiation between regular cardiac rhythms and potential irregularities detected in the audio sample. Upon identifying a feature within the sound 8G8, the algorithm then generates a descriptor for each, acting as a distinct fingerprint or identifier for that feature. This can involve methods like Mel-frequency cepstral coefficients (MFCCs) to capture the timbre and spectral shape and is referred to as descriptor extraction.

After extracting these features and their associated descriptors, they're compared against a reference database of established sound targets that are representative of various heart and lung conditions. The AI/ML algorithm endeavors to find the most congruent features between the current audio segment and these known targets 8G9. From this comparison 8G10, a target match score 8G12 is produced 8G13 and directed to a large medical data model (LMDM) AI/ML algorithm for further metrics analysis 8G14. This can swiftly lead to on-device identifications of medical concerns or emergent conditions, denoted as Edge AI or Endpoint AI. The primary aim here is to assign the identified sound match to its corresponding match score, gauging its medical significance.

The system then correlates this match to known medical histories of the patient, potentially raising emergency medical alerts, diagnostic markers, or general medical evaluations. At least one medical professional reviews and analyzes alerts, predictions, and/or recommendations within a feedback review system 8G15. Within this system, human interactions designated as favorable 8G16 or unfavorable 8G18 are highlighted for further analysis. The collected data is then periodically reviewed by a medical data analytics team, composed of physicians and data scientists, delving into health data points, which may lead to fine tuning of the AL/ML algorithms or adjusting the data collection to remove outlier data providing poor results 8G19. Additional analysis may also be used for bias testing, model improvement and correcting bias within the model 8G20. Samples with unfavorable designations are given similar attention. After the review process, the insights gathered are used to refine and train the AI/ML system for enhanced accuracy and performance.

FIG. 8H presents a schematic representation of the Empathetic Human Process Automation (EHPA) system 8H1 in the context of patient care. Upon diagnosis of a medical condition, a patient is integrated into a coordinated care program 8H2. This program employs an automated patient conversational artificial intelligence system, grounded in the principles of the EHPA system. The system is designed to interact with patients who are newly diagnosed and those with existing medical conditions, incorporating their behavioral health and self-motivation information 8H3.

After agreeing to the terms, the patient's medical conditions and associated laboratory results and assessments are integrated into their health information 8H4. This data serves as the basis for the patient's interaction with the system 8H5A-D, accessible either through an online patient portal or a medical health monitoring device 8H7. The EHPA system processes this data, providing feedback based on continuous assessments or newly added medical data 8H8A-D. For example, a patient with high low-density lipoprotein (LDL), diabetes, obesity, and hypertension might receive a prompt about elevated blood pressure, suggesting a physician contact. This messaging utilizes cognitive automation technologies, including natural language processing (NLP), natural language understanding, generative artificial intelligence (GAI), and other cognitive automation systems to deliver informative feedback and humanistic interactions that are aware and incorporate various patient information data points. The system can also provide positive reinforcements for improved health metrics, such as a decrease in LDL or an increase in high-density lipoprotein (HDL), or constructive feedback in areas needing attention 8H9A-F. Patients may have the option to provide feedback on these interactions, categorizing them as favorable or unfavorable. This feedback is used for further analysis. At least one medical professional reviews and analyzes alerts, predictions, and/or recommendations within a feedback review system 8H10. Within this system, human interactions designated as favorable or unfavorable are highlighted for further analysis. The collected data is then periodically reviewed by a medical data analytics team, composed of physicians and data scientists, delving into health data points, which may lead to fine tuning of the AL/ML algorithms or adjusting the data collection to remove outlier data providing poor results. Additional analysis may also be used for bias testing, model improvement and correcting bias within the model. Samples with unfavorable designations receive additional scrutiny. The insights from this process are used to refine the artificial intelligence and machine learning algorithms of the EHPA system. Feedback and review outcomes are channeled into the artificial intelligence and machine learning system's training, aiming for improved accuracy and performance in future interactions.

FIG. 8I is a schematic representation of modes of usage for reminders and continuous monitoring automation 8I1. First, the user may set up a health monitoring device for reminders 8I2. Next, the user selects the reminder frequency 8I3. The settings allow for daily 8I4, weekly 8I5, bi-weekly 8I6, and monthly 8I7 settings. The reminder may be a notification that reminds the person to take their health monitoring reading by a medical device capable of using biological sensors such as an ECG, pulse oximeter, temperature that may be skin or core body temperature, blood pressure, heart and lung sounds or images and videos of the skin, ear, nose, and throat. The reminder may be an alarm sound, an artificial speaking voice, haptic feedback, or a combination of all the alarm notifications capabilities. This may include notification settings that are transferred from the device to a database which may transmit the alarm notifications on other mediums.

For the daily setting the person can choose the days in the week that may enact 1 or more medical device reminders.

For the weekly setting the user may select a monthly week cycle for a month 8I9 or a range of months that may be a selection of 1 or more weeks in a month. Once the user selects the weekly cycle for the month or a range of months the user can then select the days in the week that may enact 1 or more medical device reminders 8I8.

For the bi-weekly selection the user may select 2-week cycles by choosing a week and then an additional week that skips the following week from the first week chosen. Once the user selects the bi-weekly cycle for a month or a range of months that will be 2 or more weeks in a month, the user can then select the days in the week that should enact 1 or more medical device reminders 8I9.

For the monthly selection the user may select a monthly reminder setting for a range of time where the user may select a day of the month that should enact 1 or more medical device reminders 8I10.

Once the user selects the days of the medical reminders, next the user may select the time slots in which the health monitoring device will initiate a notification 8I11. Other algorithms may be used to suggest the time of day which is best to take measurements and the medical device user may agree to the suggested day/time reminder time slots. A time-slot would indicate a time or period-of-time that would initiate a reminder. The patient may choose multiple time slots or suggested to use multiple time slots on the particular day. The time slots chosen can be for all days, a selection of days in the month or week period, or all days individually. Additionally, the device user may select 1 or more biological sensors that should be included in the scope of the current reminder setting 8I12.

Next, the user may select the modes of contact for the reminder system 8113. This may be described as the one or more ways in which the user is notified of the reminder. Contact may be initiated directly on the health monitoring medical device 8I14, by a notification such as an SMS/MMS mobile phone communication message 8I15, social notifications, such as social messaging platforms 8I17 including but not limited to WhatsApp, Facebook, or Facetime, electronic mail (E-mail) 8I16 and other notification health alert platforms such as those that would be on a patient portal or other types of web alerting systems.

The device user may then save the reminder settings and may be prompted to set up additional reminders with the same reminder settings that were previously used and/or new reminder settings to the scope of the new current settings 8I18.

In an additional set up of the health monitoring medical device a user may select settings for monitoring modes and/or continuous monitoring automation 8I19. In this process the user first selects the monitoring frequency setup setting on the medical device 8I20. Then the user may select the monitoring mode 8I21 which may be single use mode 8I22, multiple single Sensor mode (which may be for 1 or more sensors using continuous automation) 8I23 or continuous use mode 8I24.

Single use mode is the default mode that is always present on the system and is available for general use. In this way, a device user may select a biological sensor to perform a reading on the medical device. When each biological sensor reading is has taken the reading the user may choose another biological sensor reading to take or end the device interaction. When the user is taking the reading, the device transmits the health data which may be transmitted to the patient's health portal, patient data store, EMR, and/or a physician management system 8I34.

Multiple single sensor mode has the capability of continuous automation where the user selects the frequency of readings over a period of time 8I25. In this mode, the user selects the frequency of how often the biological sensor readings should be taken. For example, the user may be setup to take blood pressure readings and an ECG reading every 15 minutes 8I26 over a 12 hour time-period 8I28. In this example the frequency is a reading to be taken every 15 minutes 8I29. In an additional setting, the user selects the duration for the continuous automation settings 8I30. For example, the user may select the time-period in which the total elapsed time the device readings sensor mode should last for 8I27. In this example, the frequency is that the readings are take over a 12-hour time-period 8I28. Next, the user may select the biological sensors of the health monitoring medical device that should be included in the continuous automation mode settings of the scope of the current settings 8I31. A setting may be selected to choose which biological sensor should be utilized for each frequency session where the sensors may change depending on the session in the frequency duration 8I32.

In the multiple single sensor mode, with continuous automation, each biological sensor reading that has a result is transmitted to the patient's health portal, patient date store, EMR, and/or a physician management system 8I35. The mode can be selected to run again with the same settings as the previous session or a new continuous automation setting can be used for a subsequent monitoring session 8I33.

Continuous use mode may be selected where the mode is setup to take biological sensor readings over a period-of-time only. For example, the user may have their temperature monitored for a 12 hour period or an ECG monitored over a 1 hour period. In this mode the user selects the settings for continuous use. Next, the user selects the duration of the health monitoring session. Thereafter, the user selects 1 or more biological sensor readings that should be included in the health monitoring session.

In the continuous monitoring mode the device transmits data continuously until the session is completed 8I36. When the session is finished, the user may select to run the same session again or create a new session for a subsequent session or end the medical device interaction.

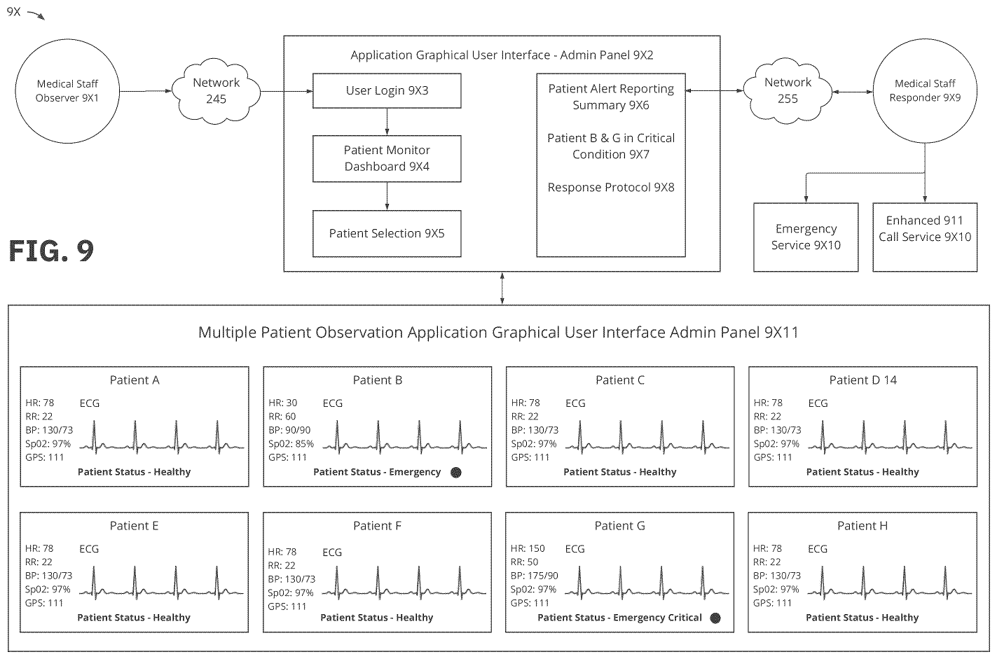

FIG. 9 is a schematic representation of an application of graphic user interface 9X2, monitoring 9X4 and notification of emergency to medical staff responder 9X1. The responder is connected to a network 245 that provides patient alert reporting summary 9X6, patient B&G in critical condition 9X7, and response protocol 9X8. The responder 9X9 is also connected to an enhanced 911 call service 9X10 to initiate emergency treatment of the patient, based upon multiple patient observation application graphic user interface administration panel.

This figure illustrates a schematic of a remote health monitoring and emergency response system for patients. Medical professionals can monitor one or more patients in real-time who need continuous observation 9X2. The purpose of this system is to replicate in-hospital monitoring capabilities from a distant location for both the medical professional and the individual being monitored. Traditional systems, typically found within hospitals, utilize equipment such as monitoring towers that continuously capture or periodically gather patient device readings. These conventional systems are typically restricted to the confines of the hospital, often to the specific floor where the patient is located. In contrast, our system performs the same monitoring functions using remote health monitoring devices, suitable for any remote setting, provided there's an internet connection available for the person being monitored.

The observer, using a device capable of connecting to a secure internet connection such as a computer, tablet, or mobile computing device, may login 9X3 into a medical monitoring administration platform. The observing member once logged into the medical monitoring platform may open a patient monitoring dashboard which has the capability of monitoring, selecting and adding new patients for the observation process 9X11.

The observation process may include applying monitoring settings for one or more persons that are currently a part of the monitoring process 9X5. Settings may include the ability to set the duration of monitoring for one or more patients. Additional settings may include the ability to set threshold parameters of what constitutes an emergency situation for one or more patients. The patient monitoring portal also has the capability of metrics and reporting on the patient health status of one or more patients. In one example, the patient monitoring dashboard has a total of 8 patients that are being monitoring and are referenced as patient A, patient B, patient C, patient D, patient E, patient F, patient G, and patient H. In this example patient B has a reported low pulse oximeter reading of 85% and should be considered an emergency. The monitoring dashboard may provide indicators that focus attention on Patient B as in need of medical attention. Another patient, patient G, has a high blood pressure and elevated heart rate with a very high temperature and should be considered of a status of a critical medical emergency.

Medical decision-tree algorithms and artificial intelligence and machine learning algorithms analyze the transmitted medical device biological sensor readings and determine the status of each patient's medical emergency which may or may not be of an emergent medical status but which may change over a period of time during the observation. Other influencing information is included in the medical device algorithms such as the patient demographics and known medical conditions, which can influence the medical alert algorithms to improve prediction accuracy, recommendations, and at-risk sensitivity correlations.

Continuing the example, the patient monitoring dashboard must alert the observing staff member that patient B and patient G have an emergent medical condition as transmitted from the medical device of each patient. The observing staff member may follow a response protocol for contacting a medical staff responder, which may be an emergency service such as a 911 emergency service, enhanced 911 (e911) call service, or direct medical staff interaction for patients that may be currently in a medical care setting such as a hospital, nursing home, in-patient care facility, out-patient care facility, or at home care.

The medical device may, for one or more patients, be equipped with a global positioning satellite (GPS) current location sensor module which may report the patients exact location within a range of accuracy compared to distance. The data may be transferred along with other biological sensor data which is transmitted with the medical device during the patient observation. This may allow the medical staff observer to report on the exact location of the patient who may be in need of medical attention. The GPS data may also allow for direct integration of the observing patient data and automatically transmit route information to an emergency service, which may be known as an e911 emergency service.

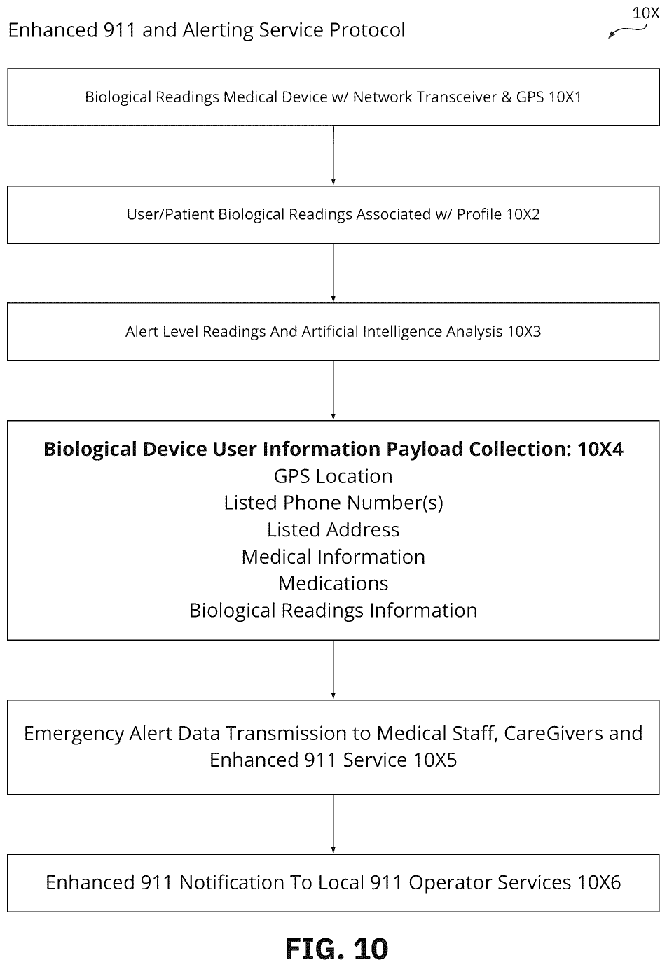

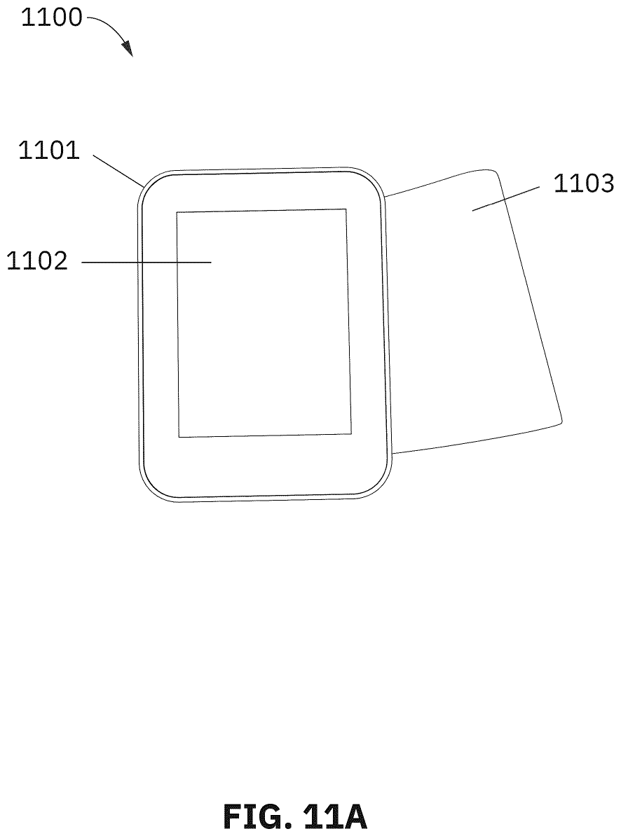

FIG. 10 is a flow diagram of an enhanced emergency and alert service protocol for transmitting medical data from a medical device 10X1 directly to an emergency service system. The medical device biological sensors may take readings 10X2 to determine whether they rise to the established alert level and are further analyzed with AI 10X3. Where a reading rises to the established alert level, user information, such as GPS location, phone number, address, medical information, medications, and biological readings information 10X4 are transmitted to medical staff, caregivers and Enhanced 911 services 10X5, which notify local 911 operator services to initiate a response by emergency personnel 10X6.

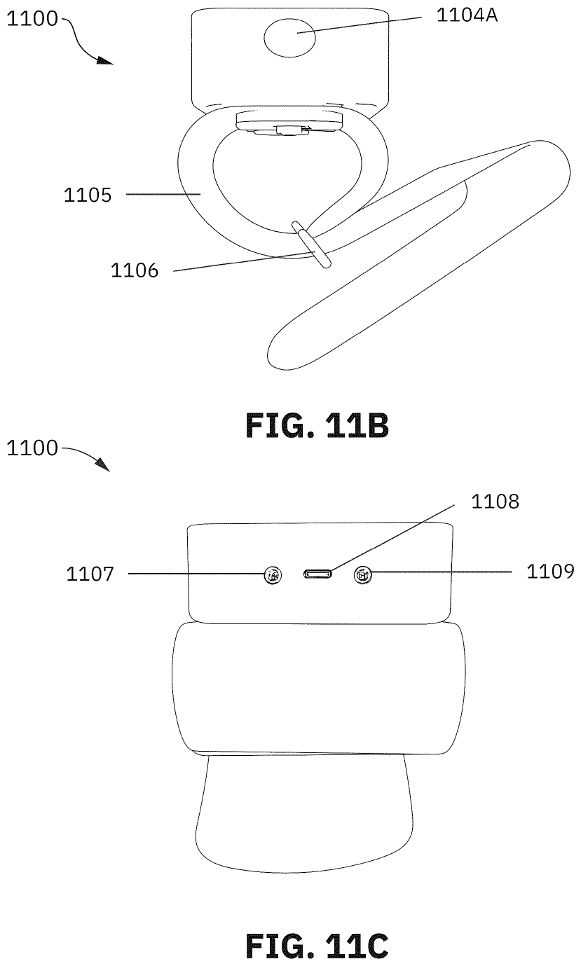

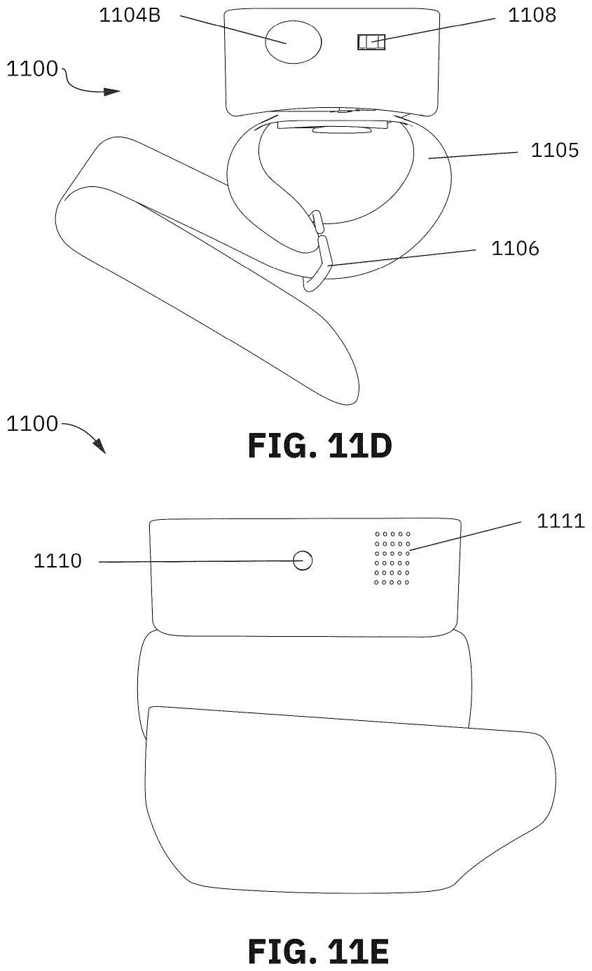

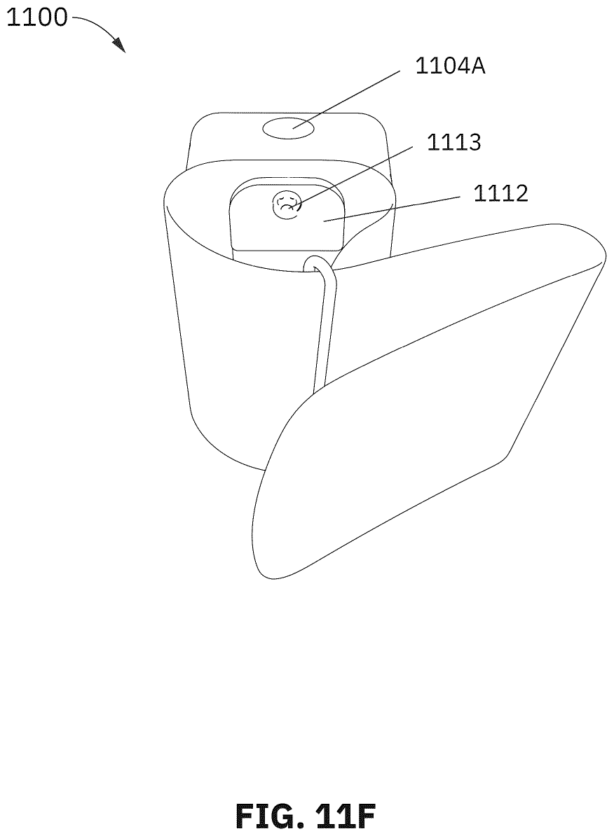

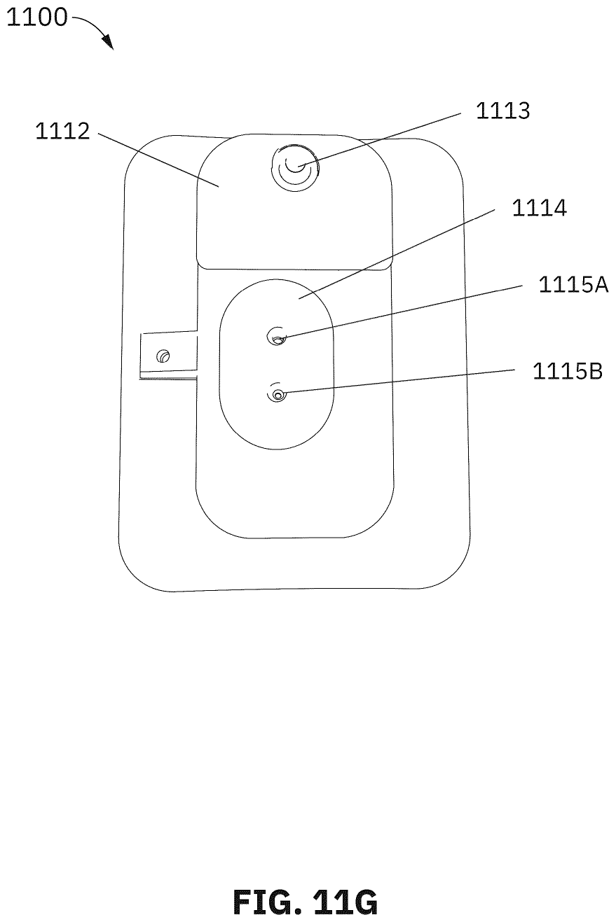

FIG. 11A discloses a top view of one embodiment of a remote health monitoring device wherein the touch screen portion 1102 of the housing component 1101 and a portion of the cuff component 1103 are visible.

FIG. 11B discloses a front view of one embodiment of the remote health monitoring device disclosed herein wherein the ECG dry sensor electrode 1104A and grounding sensor plate portions of the housing component are visible, also with the cuff 1105 and cuff ring 1106 components located below the housing component.

FIG. 11C discloses a left side view of one embodiment of the remote health monitoring device disclosed herein wherein a pulse oximeter port 1107, USB C port 1108, and gel based electrode port 1109 portions of the housing component are visible. The cuff component is also visible below the housing component.

FIG. 11D discloses a back view of one embodiment of a remote health monitoring device in accordance with one embodiment of the present invention disclosing a second ECG dry sensor electrode 1104B, pulse oximeter input 1108, blood pressure cuff 1105 and cuff ring 1106.

FIG. 11E discloses a right side view of one embodiment of the remote health monitoring device disclosed herein wherein the multifunction button 1110 and air ventilation 1111 portions of the housing component are visible.

FIG. 11F is perspective view disclosing an ECG dry sensor electrode 1104A, grounding plate 1112, temperature sensor 1113, blood pressure cuff and cuff ring.

FIG. 11G bottom view of one embodiment of the housing component of a remote health monitoring device wherein the air bladder stabilization plate 1114 and a first 1115A and second 1115B tube connector are visible as well as grounding electrode plate 1112 and temperature sensor 1113 are visible.

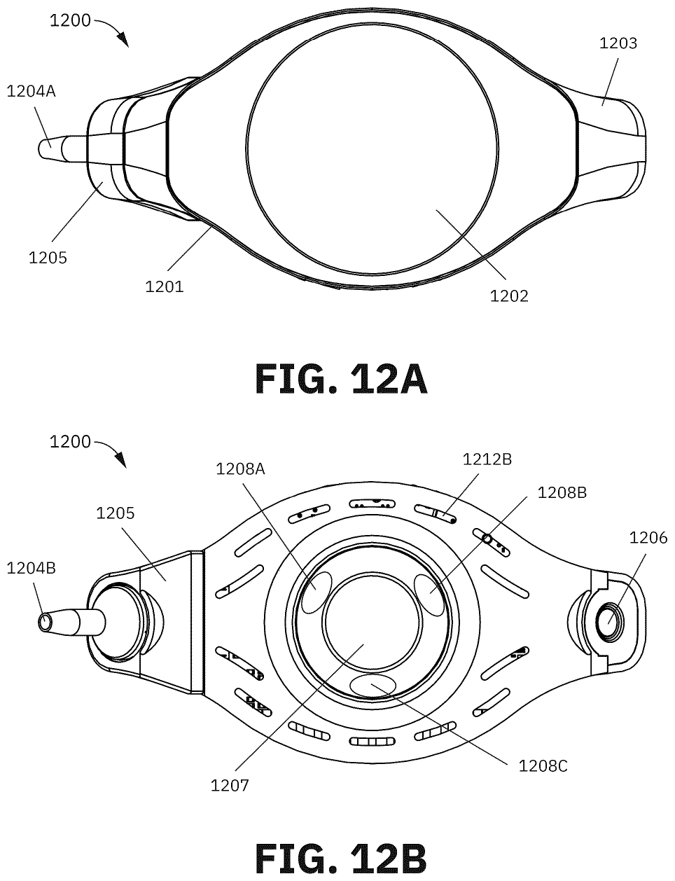

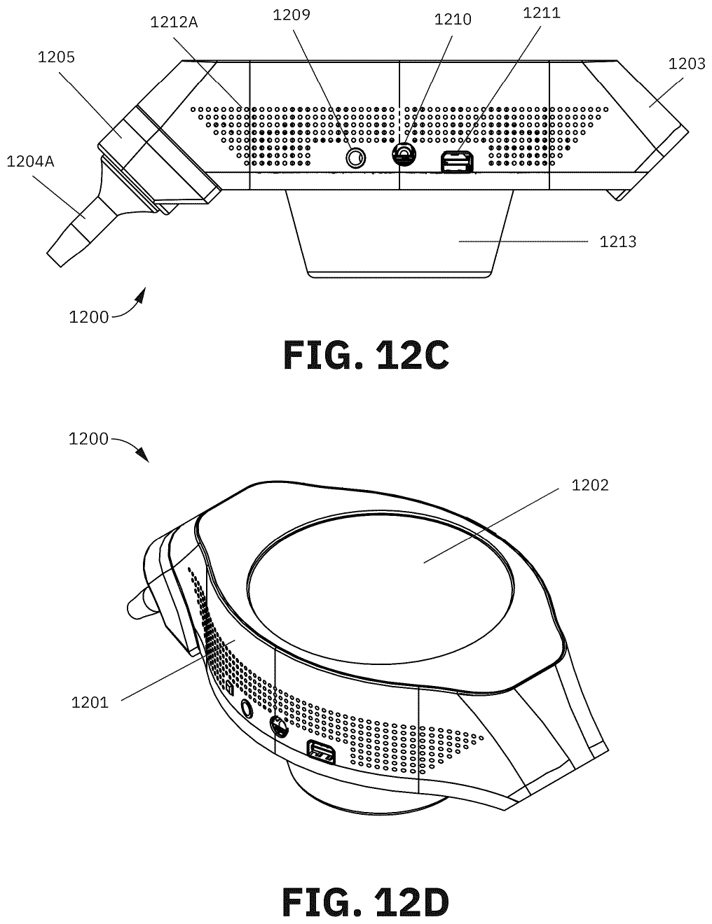

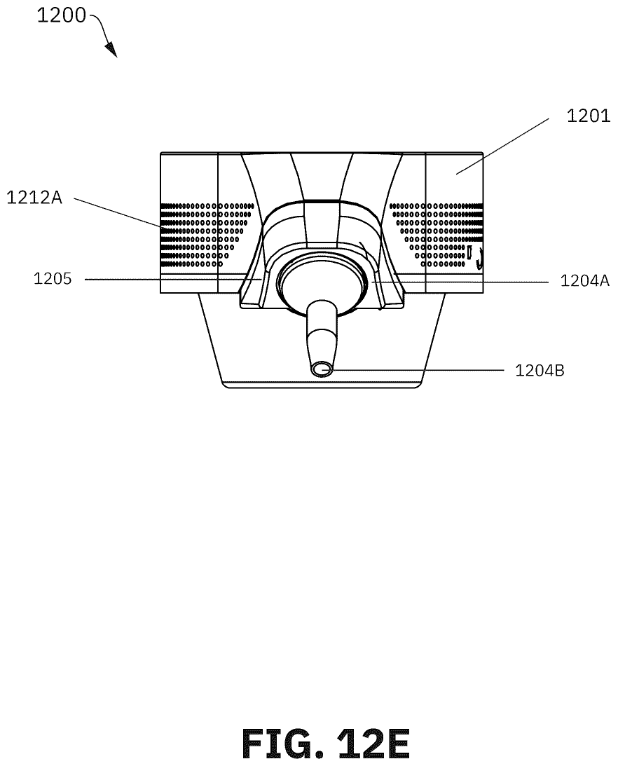

FIG. 12A is a top view of a biological observation and data collection device, in accordance with one embodiment of the present invention. This figure discloses a housing component 1201 enclosing device circuitry, power cell units, and device sensors; a touch screen component 1202; a microphone component 1203; a speculum component 1204A; and camera enclosure 1205.

FIG. 12B is a bottom view of a biological observation and data collection device, in accordance with one embodiment of the present invention. This figure discloses a digital stethoscope sensor 1207, temperature sensor 1206, speculum 1204B, air ventilation 1212B, dry electrode sensors 1208A, 1208C and grounding sensor 1208B component.

FIG. 12C is a side view of a biological observation and data collection device, in accordance with one embodiment of the present invention. This figure discloses speculum 1204A with camera sensor enclosure 1205 for visualization, display port 1211, microphone jack 1209, USB C port 1210, temperature sensor 1203, transducer housing 1213, air ventilation 1212A, and transducer housing that may include one more piezo electric elements or microphones 1213.

FIG. 12D is a perspective view of a biological observation and data collection device, in accordance with one embodiment of the present invention. This figure discloses a piezo microphone, speculum with camera sensor enclosure for visualization, reset button, microphone jack, USB C port, housing 1201 and touch screen display 1202 components.

FIG. 12E discloses a front view of one embodiment of the biological observation and data collection device. This figure discloses the base of the speculum component 1204A, opening of speculum which allows camera sensor to receive video or data 1204B, camera component 1205, and air ventilation 1202.

To further enhance the clarity and precision of the electrical signal, the apparatus integrates an additional grounding electrode. This grounding electrode, located at the base of the enclosure, plays a dual role. Not only does it ensure that the electrical signal remains clean and free from interference, but it also serves as a securing mechanism for the attached blood pressure cuff. The first and second transducer electrodes utilize conductive-plated contacts, ensuring robust and enhanced ECG data acquisition.

FIG. 13 discloses one embodiment of a biosticker disclosed herein comprising a piezo electric sensor, gyroscope, accelerometer, temperature sensor, and blue tooth network transceiver micro controller component.

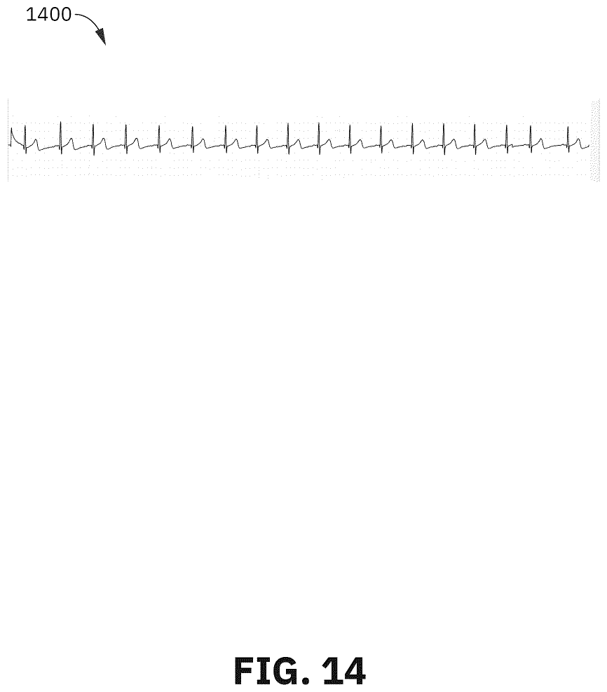

FIG. 14 is an ECG data transmission directly from the remote health monitoring device disclosed herein to a receiving device.

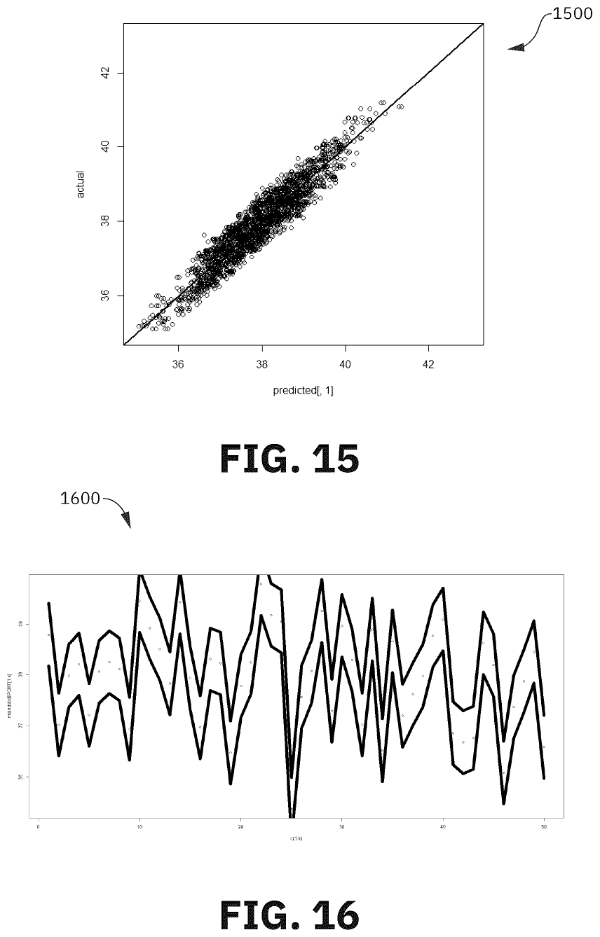

FIG. 15 is a plot point graph showing actual versus predicted plot points over time, highlighting the model's accuracy core body temp prediction from a machine learning algorithm.

FIG. 16 discloses a variation of a Bland-Altman plot line, which shows the acceptable ranges of variables that are incorporated into the model. Where actual readings fall between the predicted range, the non-linear polynomial regression calculation is robust. It is used to determine the variables are used to predict within predicted range.

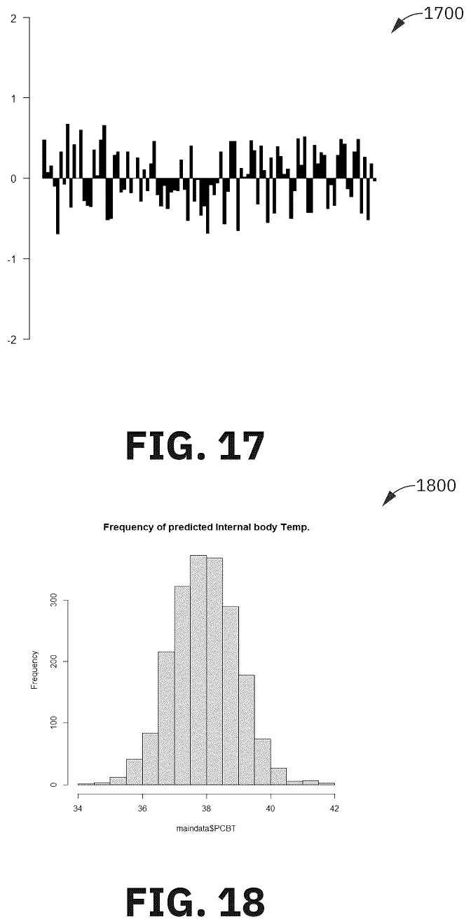

FIG. 17 This figure illustrates a plot of deleted (or externally) studentized residuals for predicted core body temperatures. The Y-axis represents the studentized residuals, which are the differences between the observed temperatures and the values predicted by the model, scaled by an estimate of their standard deviation. These residuals provide a normalized measure, making it easier to identify potential outliers or points that do not follow the expected pattern. The X-axis enumerates the predicted core body temperatures, spanning from 34° C. to 42° C. Oscillating lines above and below the zero mark on the Y-axis indicate the range of residuals for each predicted temperature. Points closer to the zero line suggest that the model's predictions are more accurate for those observations, while points further away indicate larger discrepancies between the predicted and observed values. The plot aids in assessing the model's performance across different temperature predictions and identifying areas where the model might be improved.

FIG. 18 presents a histogram illustrating the frequency distribution of predicted core body temperatures by the model. The X-axis represents the range of predicted temperatures, spanning from 34° C. to 42° C. The Y-axis denotes the frequency of each predicted temperature. A prominent peak is observed at 38° C. with a frequency of 300, indicating that this temperature is the most commonly predicted value by the model. This peak suggests that the model has a high confidence in predicting temperatures around the 38° C. mark, potentially due to its prevalence in the training dataset. The distribution provides insights into the model's prediction tendencies and areas of high confidence.

The schematic representation of the Biological Sensor Reading Prediction (BSRP) system is depicted in FIGS. 4, 5, and 6. This system is designed to predict biological sensor readings, such as core body temperature, by leveraging both traditional machine learning models and advanced deep learning techniques.

Initially, the system gathers data from various sources, including wearable devices, ambient temperature sensors, and user inputs. This data encompasses a range of variables, from environmental factors to individual physiological metrics. The primary aim is to understand the relationships between these variables and the target sensor reading.

On the device, various models such as non-linear regression, random forest, support vector machines, decision trees, or a lite version of a deep neural network, which may be referred to as Edge AI or Endpoint AI, are employed. These models, trained on a subset of the data, are optimized for immediate predictions. The model may be represented by variables, action functions, coefficients.

In parallel, the Large Medical Data Model (LMDM), a deep learning neural network, resides on a central server. This model is trained on a more extensive dataset, encompassing a broader range of variables and more complex relationships. The LMDM is particularly adept at capturing non-linearities and interactions between variables that might be challenging for traditional models.

Iterative refinement is a cornerstone of the BSRP system. As more data is collected on the edge device, it can be sent back to the central server periodically. This data is used to retrain or refine the LMDM. Once the LMDM is updated, a new version of the local model can be deployed back to the edge devices, benefiting from the insights gained from the deep learning model.

This approach combines the best of both worlds: real-time predictions on the edge and the ability to improve the model over time using new data. The system ensures that predictions are not only immediate but also continuously refined, ensuring the highest accuracy possible.

The insights from this process are used to refine both the local models and the LMDM. Feedback and review outcomes are channeled into the training of both models, aiming for improved accuracy and performance in future predictions.

Referring to the accompanying diagrams, various graphical representations illustrate the performance and relationships captured by the models. One graph showcases actual versus predicted plot points over time, highlighting the model's accuracy.

In one embodiment, a wearable remote biomonitoring device is disclosed herein that enables practitioners or caregivers to remotely monitor patients. The device comprises at least a pulse oximeter, heart rate sensor, blood pressure sensor, temperature sensor, electrocardiogramonitor, accelerometer, air pump, gyroscope, speaker, microphone. Auxiliary devices such as an accelerometer, stethoscope, otoscope, gyroscope, chest straps, bio-stickers, or wet/dry electrodes to collect data for respiration, heart rhythm, or electrocardiogramay be coupled to the device for collecting additional data streams without the need for a wireless internet connection. Chest straps and bio-stickers may also be coupled using Bluetooth technology for collecting data related to a patient's respiratory rate.

In another embodiment of the present invention, a remote data collection device is disclosed for a obtaining a snapshot of a patient's condition, including the patient's temperature, or observing the patient's ear, nose, throat, eyes, or skin for abnormalities, just as one would during an in-person doctor's visit. The device allows for remote manipulation of the camera by a practitioner so that a more comprehensive examination can be conducted. Once the camera is in position for examination, the provider may manipulate the view of the camera to inspect areas of particular interest.

In an example embodiment, an apparatus for medical applications is provided. The apparatus comprises one or more modules for collecting and processing biological data. These modules are housed within a protective casing.

The apparatus also includes a system for managing heat within the apparatus. This system is also housed within the protective casing.

The apparatus further comprises a module for data processing and execution of instructions. This module is configured to record the patient's biological data and execute on-device machine learned artificial intelligence, computer vision for video and image data analysis, and at least one artificial intelligence algorithm for audio data analysis.

The apparatus also includes a module for wireless communication. This module is configured to communicate with one or more remote data storage and processing units for receiving in real-time biological data.

In another embodiment, the one or more modules for collecting and processing biological data include an imaging device for capturing biological image and video data, an audio capturing device for capturing biological auscultation sound data, a device for measuring temperature, a motion detection device for detecting data related to a person's movement, a device for detecting axial position, a device for processing temperature data, a module for handling video data, and a module for handling audio data.

In yet another embodiment, the system for managing heat within the apparatus includes a component for heat conduction connected to one or more processing units for data analysis utilizing a material for heat conduction, a component for heat dissipation, and a system for air movement to displace hot air from inside of the protective casing to outside of the protective casing.

In a further embodiment, the module for data processing and execution of instructions is implemented by one or more data processing units configured with instructions in a permanent storage medium, that when executed cause the one or more data processing units to record the patient's biological data and execute on-device machine learned artificial intelligence, computer vision for video and image data analysis, and at least one artificial intelligence algorithm for audio data analysis.

In another embodiment, the apparatus for medical applications further comprises a visual output interface for displaying visual data. This visual output interface is at least partially surrounded by a protective casing.

In yet another embodiment, the apparatus for medical applications further comprises an audio output device for outputting audio data. This audio output device is at least partially surrounded by a protective casing.

The present disclosure relates to a medical device that can be worn for monitoring of a person's vital signs. In an example embodiment, the device comprises a housing, a display at least partially surrounded by the housing, and at least one component for gathering a person's data that is at least partially surrounded by the housing.

The components for gathering a person's data may include a thermometer component for monitoring a person's temperature, a pulse oximeter component for monitoring a patient's oxygen levels, an electrocardiogram component for monitoring electrical signals from the person's heart, an accelerometer for monitoring the person's movement, a gyroscope for monitoring a person's positional axis, a sphygmomanometer for monitoring a patient's blood pressure, a global positioning system, a speaker component, a network transceiver for sending and receiving data transmissions, at least one port for inputting at least one adaptor for connecting an additional medical component, a computing component implemented by one or more processors configured with instructions in a non-transitory memory, that when executed cause the one or more processors to record the patient's biological data and execute on-device machine learned artificial intelligence, and a transmitting component for wirelessly communicating with at least one remote server for receiving in real-time biological data from one or more components of the medical device.

In another example embodiment, the medical device may further comprise at least one Bluetooth enabled secondary device, at least one port enabled secondary device, an interactive touchscreen display, a computing component configured to execute an on-device machine learned artificial intelligence program to make a health or biological sensor reading prediction.

In yet another example embodiment, the medical device may comprise a display at least partially surrounded by a housing, at least one component for gathering biological data and processing information that is at least partially surrounded by the housing, including a camera sensor for collecting biological image and video data, a microphone sensor for collecting biological auscultation sound data, a temperature sensor for collecting a patient's temperature data, a speaker component, an accelerometer component for detecting data related to a person's movement, a gyroscope component for detecting a person's axial positional data, a thermometer component for processing a person's temperature data, a video processing component for processing a person's biological video data, an audio processing component for processing a person's biological audio data, a thermal transfer system comprising a thermal transfer plate connected to the CPU and GPU modules utilizing a thermal transfer substance, a thermal transfer heat sink, and an air circulatory system to displace hot air from inside of the enclosure to outside of the enclosure, a computing component implemented by one or more processors configured with instructions in a non-transitory memory, that when executed cause the one or more processors to record the patient's biological data and execute on-device machine learned artificial intelligence, computer vision for video and image data analysis, at least one artificial intelligence algorithm for audio data analysis, a central processing unit and a graphics processing unit for analysis of patient data on the medical device, and a transmitting component for wirelessly communicating with at least one remote server for receiving in real-time biological data from one or more components of the medical device.

In another example embodiment, the medical device may further comprise at least one protocol for transmitting data directly from the device to a remote server; the data transmission architecture comprised of an event driven data network that utilizes publish and subscribe communication protocols.

In yet another example embodiment, the medical device may further comprise a machine learned artificial intelligence algorithm for predicting sensor readings or a medical event based on readings derived from at least one sensor, a supervised machine learned artificial intelligence program for predicting sensor readings or a medical event based on readings derived from at least one sensor, and analysis of data from sources other than a sensor, for predicting a medical event.

In another example embodiment, the medical device may comprise a machine learned deep learning neural network comprised of supervised training data sources, and unsupervised training data sources affecting medical information culminated from more than one person, which may include medical device data and is composed of 3 or more deep learning algorithms for generating a large medical data model for comprehensive data analysis and artificial intelligence applications used directly on medical devices that contain CPU's and GPU's or through data transferred to remote servers that consist of CPU's and GPU's.

In yet another example embodiment, the medical device may further comprise a machine learned deep learning neural network comprised of supervised training data and unsupervised training data sources composed of large medical data models that can be used for unsupervised learning characteristics such as clustering data based on K-means for the purposes of grouping, categorizing and identifying medical data, analyzing data relationships among variables in various data sets for the purposes of improving medical outcomes, and analyzing data associations to determine the usefulness and removal of data relationships that are not helpful in improving medical outcomes.

In another example embodiment, the medical device may further comprise a semi-supervised machine learning method for combining supervised machine learning with unsupervised machine learning to further classify unsupervised data for more accurate predictability by training the unsupervised machine learned data with human assisted data corrections for a model that includes precision data analysis from an unsupervised machine learning data set.

In yet another example embodiment, the medical device may further comprise a method of providing coordinated patient care, the method comprising obtaining patient medical data, including data transmitted directly from a medical device, transmitting and storing the patient medical data in a patient-controlled data store, permitting a medical provider access to the patient medical data, wherein the medical provider may obtain the patient medical data using a patient management system and may generate a medical record for storage in the patient-controlled data store in real time from more than one provider, and granting access by the patient to at least one provider to the patient's data for coordinating patient care.

In another example embodiment, the medical device may further comprise a machine learning deep learning neural network comprised of supervised and unsupervised data sources affecting medical information for more than one person for providing information to large platform data analysis in order to make a prediction or an assertion from clustering of data.

In yet another example embodiment, the medical device may further comprise semi-supervised machine learning for a model that is comprised of both unsupervised and supervised data sets whereby a person trains and tags data from a review of predicted data results of one or more data predictions.

In another example embodiment, the medical device may further comprise a method for training supervised machined learning data by examining the results of a predicted health alert recommendation and transmitting the approval or correction to a large machine learning deep neural network algorithm, and using that new algorithm training for improved artificial intelligence model performance.

In yet another example embodiment, the medical device may further comprise a system for automating medical care interactions, the system comprising a human process cognitive automation artificial intelligence model providing a patient with an automated interaction relating to medical and personal health information, providing the patient with a communication based on the patient's medical data, the communication being either by text or verbal human sounding voice interactions, visual ques, sounds, or vibrations, and the communication providing the patient with medical education, health recommendations, current medical analysis, health alerts, or a possible future medical event, wherein the cognitive automation is based on artificial intelligence generated from medical data belonging to more than one person.

In another example embodiment, the medical device may further comprise a continuous artificial intelligence training feedback mechanism, wherein data collected is transferred to an artificial intelligence data analytics server to further improve the data analytics model, and continuously train a large machine learning deep neural network algorithm, wherein an application using the artificial intelligence algorithm is continuously updated to a newer and improved machine learning artificial intelligence model.

In yet another example embodiment, the medical device may further comprise human process cognitive automation, wherein at least one interaction is initiated by a signal that is generated from data analyzed by artificial intelligence.

In another example embodiment, the medical device may further comprise human interaction recommendations automated by at least one artificial intelligence algorithm based on at least one trigger from deep learning and artificial intelligence algorithms, including notifications, email, SMS, and artificial intelligence driven chatbot dialog interactions.

In yet another example embodiment, the medical device may further comprise a scored interaction, wherein each interaction is weighted, including a dialog driven automated cognitive interaction that scores information gathered from an interaction, including from a questionnaire, wherein the scoring mechanism determines whether an interaction is a non-concern, concern, or emergent concern, and whether the interaction is classified as a compliant or non-compliant interaction.

In another example embodiment, the medical device may further comprise a person's personal information, medical information, mental health information and wellbeing information to provide a cognitive interaction, which may include medical education and information to the patient for the purpose of collecting information for a machine learning model, a medical caretaker, and to provide therapeutic communications and interactions to the person.

In yet another example embodiment, the medical device may further comprise feeding data back to the human process cognitive automation model, that can include provider supervised training.

In another example embodiment, the medical device may further comprise permitting access to the patient's electronic medical record to at least one medical provider.

In yet another example embodiment, the medical device may further comprise feeding data back to the human process cognitive automation model, that can include human supervised training.

In another example embodiment, the medical device may further comprise permitting access to data by the patient to third parties, including family members, research studies, or medical personnel.

In yet another example embodiment, the medical device may further comprise analysis of the patient's data by a human or artificial intelligence.

In another example embodiment, the medical device may further comprise a method of providing coordinated patient care is single use patient monitoring.

In yet another example embodiment, the medical device may further comprise a method of operating the device, wherein an observational mode is selected from the group comprising: single use mode, intermittent mode, or continuous mode, wherein single use mode comprises obtaining one or more sensor readings at any desired point in time; intermittent mode comprises obtaining one or more sensor readings at a desired time interval over a fixed period of time, the selected sensors being predetermined prior to commencement of the observational period; and a continuous mode comprising a continuous operation of one or more sensors for obtaining sensor readings over an observational period.

In another example embodiment, the medical device may further comprise a method of operation of the device, wherein an observational mode is selected from the group comprising: single use mode, intermittent mode, or continuous mode, wherein single use mode comprises obtaining one or more sensor readings at any desired point in time; intermittent mode comprises obtaining one or more sensor readings at a desired time interval over a fixed period of time, the selected sensors being predetermined prior to commencement of the observational period; and a continuous mode comprising a continuous operation of one or more sensors for obtaining sensor readings over an observational period, wherein data may be transmitted in real-time to a remote server for storage of the data in a patient data store.

In yet another example embodiment, the medical device may further comprise a method of operation of the device, wherein an observational mode is selected from the group comprising: single use mode, intermittent mode, or continuous mode, wherein single use mode comprises obtaining one or more sensor readings at any desired point in time; intermittent mode comprises obtaining one or more sensor readings at a desired time interval over a fixed period of time, the selected sensors being predetermined prior to commencement of the observational period; and a continuous mode comprising a continuous operation of one or more sensors for obtaining sensor readings over an observational period, wherein data is transmitted to a patient data store or a general data store, and shared with a permitted observer in real-time for remote patient monitoring, the permitted observer having obtained access to the patient data from a device administrator.

In another example embodiment, the medical device may further comprise a method of operation of a medical device wherein the device can be operated remotely to obtain at least one biological sensor reading, the method comprising granting access to a remote operator by a device administrator, granting permission to the remote operator to control the device, control of the device may include directing a sensor to obtain at least one biological sensor reading, or selecting a mode of operation for an observational period, other than single use mode, granting permission to the remote operator for a specified period of time, initiating a data transmission by the remote operator of at least one sensor reading, and transmitting and storing data to the remote operator through a secure connection, wherein data is encrypted in transit and at rest.

In yet another example embodiment, the medical device may further comprise a method of transmitting biological data from a device to a patient data store on a remote server, the method comprising obtaining data from a sensor on the device, transferring data from the sensor to a network transceiver on the device, transmitting data from the network transceiver to a remote server, using a secure and encrypted connection, and storing the data in a patient data store.

In another example embodiment, the medical device may further comprise a method of transmitting biological data from a device to an observer in real time, the method comprising adding an observer profile to a medical device data store, granting permission to the observer to access historical and/or real-time data, wherein the permission granted may be ongoing or for a single session, obtaining data from a sensor on the device, transferring data from the sensor to a network transceiver on the device, transmitting data from the network transceiver to a remote server, using a secure and encrypted connection, and publishing the data to a real-time streaming data store and a historical data store, wherein the data can viewed by the observer from a computing device.

In yet another example embodiment, the medical device may further comprise alerting the observer that there is an emergent medical event based on data analysis, algorithmic data analysis, or artificial intelligence analysis.

In another example embodiment, the medical device may further comprise a method wherein artificial intelligence on the medical device is utilized to improve sensor accuracy.

In yet another example embodiment, the medical device may further comprise a method wherein artificial intelligence on the medical device is utilized to predict at least one sensor reading.

In another example embodiment, the medical device may further comprise a method of providing coordinated patient care by collecting, analyzing, and applying artificial intelligence to patient data, the method comprising obtaining patient medical data, including data obtained directly from a medical device, demographic patient data, behavioral health, mental health, and data found in a medical health record, and transmitting the patient's data to a machine learning artificial intelligence data model, wherein a person's data is applied against the machine learning artificial intelligence data model to generate conclusions based on the patient's information, such as patient summarization of medical information, recommendations of care, predictive heath outcomes, and cognitive automated patient interactions.

In yet another example embodiment, the medical device may further comprise a method wherein data obtained directly from a medical device is used to continuously train and improve an artificial intelligence machine learning model used for predicting and analyzing patient data.

In another example embodiment, the medical device may further comprise a method wherein patient data gathered from a medical device and/or a source other than the medical device is used to create a comprehensive patient data model for predicting and analyzing patient information.

In yet another example embodiment, the medical device may further comprise a method wherein more than one patient is identified for remote observation and has granted permission to the observer, biological readings data, including audio and video data streams, are available to the observer in real time, the user interface for the observer is a centralized observation dashboard for remote monitoring of patient data, and alerting the observer that there is an emergent medical event based on data analysis, algorithmic data analysis, or artificial intelligence analysis.

In another example embodiment, the medical device may further comprise a method wherein the device conducts a health risk analysis utilizing algorithms and artificial intelligence to recognize an emergent event and automatically contact an emergency medical service and/or an enhanced 911 services, notifies the service of the emergent event, provides the GPS location of the device to the service, and transmits demographic information and data that is monitored and collected by the device, utilizing the network transceiver component of the invention.

In yet another example embodiment, the medical device may further comprise a method of notifying a permitted third party of a medical event from a medical device, the method comprising initiating observation mode on the medical device, identifying an emergent event utilizing sensor algorithms and artificial intelligence from one or more sensors, and notifying the permitted third party of the likely emergent event.

In another example embodiment, the medical device may further comprise a method of transmitting a notification to an emergency medical service, identifying the likely medical event, providing GPS location coordinates of the device to the emergency medical service, delivering patient demographic information, such as the patient's name, address, and known medical information, including sensor reading data to the emergency medical service, confirming receipt of the transmission, and initiating a response by the emergency medical service.

In yet another example embodiment, the medical device may further comprise a method of providing coordinated patient care, the method comprising accessing patient data in the patient-controlled patient data store, by a medical provider, wherein the medical device data can be transmitted and viewed by the medical provider in real-time, conducting a virtual remote telehealth session between the patient and the medical provider, wherein the medical provider can assess the data in the patient data store during the telehealth visit to support a medical diagnosis and treatment plan, and providing an update to the electronic medical record, which may include a transcript of the telehealth visit, notes regarding the visit, a medical order, and/or a diagnosis and treatment plan to the patient data store in real-time.

In another example embodiment, the medical device may further comprise a method of operating the medical device remotely to obtain one or more biological sensor readings in association with a virtual remote telehealth session, wherein the medical provider may assist the patient in taking a biological sensor reading remotely, and the medical provider may visually verify that the reading taken and the data transmitted to the provider is that of the patient associated with the virtual visit.

In yet another example embodiment, the medical device may further comprise a method of providing access to real-time and historical patient data from the medical device during a virtual remote telehealth session.

In another example embodiment, the medical device may further comprise a method of predicting a patient's current or future medical condition utilizing artificial intelligence directly on the device or a machine learning data analytics server.

In yet another example embodiment, the medical device may further comprise a medical device with cognitive automation capabilities, including artificial intelligence programed on the device, that interacts with the patient directly to provide patient feedback, based upon medical information obtained from one or more sensors on the device or medical information recorded onto the device, to generate cognitive interactions directly on the device to elicit a positive, concerning, and/or alerting type communication to the patient.

In another example embodiment, the medical device may further comprise a continuous artificial intelligence training feedback mechanism on a medical device where data collected on the device is transferred to an artificial intelligence data analytics server, consisting of data from interactions with the patient, the data collected on the device from patient interactions is transmitted to a data analytics server to further train and improve the data analytics model used on the device, wherein data consisting of more than one patient's information and is used to train a large machine learning deep neural network algorithm, wherein the artificial intelligence algorithm on the device is updated to a newer and improved machine learning artificial intelligence model.

In yet another example embodiment, the medical device may further comprise a method of providing health feedback to the patient regarding the patient's medical status, based on one or more patient informational data streams, including data from a medical device, and initiating a communication to the patient regarding a change to the patient's medical status.

In another example embodiment, the medical device may further comprise a method of data transmission for real time medical biological readings obtained directly from a medical device, the method comprising connecting a medical device to the internet through a network transceiver on the medical device, the transceiver protocols on the medical device provide for continuous publishing and subscribing of biological medical data, and transmitting the biological medical data from the medical device network transceiver to a receiving device through an internet connection.

In yet another example embodiment, the medical device may further comprise a method wherein biological medical data is transmitted from a medical device to a receiving device through a peer-to-peer connection, the method further comprising selecting the data recipient on the medical device, the device initiating a biological data reading, and transfers data through a secured peer-to-peer connection, and connecting the receiving device to the medical device through a secured connection that transmits data in real-time.

In another example embodiment, the medical device may further comprise a method wherein biological medical data is transmitted from a medical device to a receiving device through an intermediate server, the method further comprising selecting the data recipient on the medical device, the device initiating a biological data reading, and transfers data through a secured connection to an intermediate server, and connecting the receiving device to the routing server through a secured connection that transmits data in real-time.

In yet another example embodiment, the medical device may further comprise a method for synchronizing data on a receiving device, the method comprising collecting data on a medical device, choosing a remote data store or a receiving device data store, on the medical device, upon establishing internet connectivity, sending data not previously transmitted directly to the remoted data store or receiving device data store, and synching the data on the remote data store or receiving device data store with data on the medical device.

1. A medical device that can be worn for monitoring of a person's vital signs, the device comprising:
a housing;
a display, at least partially surrounded by the housing;
at least one component for gathering a person's data that is at least partially surrounded by the housing, including:
a thermometer component for monitoring a person's temperature;
a pulse oximeter component for monitoring a patient's oxygen levels;
an electrocardiogram component for monitoring electrical signals from the person's heart;
an accelerometer for monitoring the person's movement;
a gyroscope for monitoring a person's positional axis;
a sphygmomanometer for monitoring a patient's blood pressure;
a global positioning system;
a speaker component;
a network transceiver for sending and receiving data transmissions;
at least one port for inputting at least one adaptor for connecting an additional medical component;
a computing component implemented by one or more processors configured with instructions in a non-transitory memory, that when executed cause the one or more processors to record the patient's biological data and execute on-device machine learned artificial intelligence; and
a transmitting component for wirelessly communicating with at least one remote server for receiving in real-time biological data from one or more components of the medical device.

2. The medical device disclosed in claim 1, further comprising at least one Bluetooth enabled secondary device.

3. The medical device disclosed in claim 1, further comprising at least one port enabled secondary device.

4. The medical device disclosed in claim 1, further comprising an interactive touchscreen display.

5. The medical device disclosed in claim 1, further comprising a computing component configured to execute an on-device machine learned artificial intelligence program to make a health or biological sensor reading prediction.

6. A medical device comprising:
a display at least partially surrounded by a housing;
at least one component for gathering biological data and processing information that is at least partially surrounded by the housing, including:
a camera sensor for collecting biological image and video data;
a microphone sensor for collecting biological auscultation sound data;
a temperature sensor for collecting a patient's temperature data;
a speaker component;
an accelerometer component for detecting data related to a person's movement;
a gyroscope component for detecting a person's axial positional data;
a thermometer component for processing a person's temperature data;
a video processing component for processing a person's biological video data;
an audio processing component for processing a person's biological audio data;
thermal transfer system comprising a thermal transfer plate connected to the CPU and GPU modules utilizing a thermal transfer substance, a thermal transfer heat sink, and an air circulatory system to displace hot air from inside of the enclosure to outside of the enclosure;
a computing component implemented by one or more processors configured with instructions in a non-transitory memory, that when executed cause the one or more processors to record the patient's biological data and execute on-device machine learned artificial intelligence, computer vision for video and image data analysis, at least one artificial intelligence algorithm for audio data analysis;
a central processing unit and a graphics processing unit for analysis of patient data on the medical device; and
a transmitting component for wirelessly communicating with at least one remote server for receiving in real-time biological data from one or more components of the medical device.

7. The medical device disclosed in claim 6, wherein live-streaming data may be viewed simultaneously on a remote receiving device and, either an onboard display, or a port-connected display.

8. The medical device disclosed in claim 6, wherein the information processed is a data point.

11. The medical device disclosed in claim 1 or claim 6, further comprising at least one protocol for transmitting data directly from the device to a remote server; the data transmission architecture comprised of an event driven data network that utilizes publish and subscribe communication protocols.

12. The medical device disclosed in claim 6, wherein audio or visual information is simultaneously streamed on a remote monitoring device and the display component of the medical device for real time observation and analysis of the person's biological audio and/or visual information.

13. The medical device disclosed in claim 6, wherein audio or visual information is simultaneously streamed on a remote monitoring device and a local monitoring device for real time observation and analysis of the person's biological audio and/or visual information.

14. The medical device disclosed in claim 1 or claim 6, further comprising a machine learned artificial intelligence algorithm for predicting sensor readings or a medical event based on readings derived from at least one sensor.

15. The medical device disclosed in claim 1 or claim 6, further comprising a supervised machine learned artificial intelligence program for predicting sensor readings or a medical event based on readings derived from at least one sensor; and
analysis of data from sources other than a sensor, for predicting a medical event.

16. A machine learned deep learning neural network comprised of supervised training data sources; and
unsupervised training data sources affecting medical information culminated from more than one person, which may include medical device data and is composed of 3 or more deep learning algorithms for generating a large medical data model for comprehensive data analysis and artificial intelligence applications used directly on medical devices that contain CPU's and GPU's or through data transferred to remote servers that consist of CPU's and GPU's.

17. The method disclosed in claim 16, further comprising a machine learned deep learning neural network comprised of supervised training data and unsupervised training data sources composed of large medical data models that can be used for unsupervised learning characteristics such as clustering data based on K-means for the purposes of grouping, categorizing and identifying medical data;
analyzing data relationships among variables in various data sets for the purposes of improving medical outcomes; and
analyzing data associations to determine the usefulness and removal of data relationships that are not helpful in improving medical outcomes.

18. The method disclosed in claim 16, further comprising a semi-supervised machine learning method for combining supervised machine learning with unsupervised machine learning to further classify unsupervised data for more accurate predictability by training the unsupervised machine learned data with human assisted data corrections for a model that includes precision data analysis from an unsupervised machine learning data set.

19. A method of providing coordinated patient care, the method comprising:
obtaining patient medical data, including data transmitted directly from a medical device;
transmitting and storing the patient medical data in a patient-controlled data store, permitting a medical provider access to the patient medical data, wherein the medical provider may obtain the patient medical data using a patient management system and may generate a medical record for storage in the patient-controlled data store in real time from more than one provider; and
granting access by the patient to at least one provider to the patient's data for coordinating patient care.

20. The method disclosed in claim 19, further comprising a machine learning deep learning neural network comprised of supervised and unsupervised data sources affecting medical information for more than one person for providing information to large platform data analysis in order to make a prediction or an assertion from clustering of data.

21. The method disclosed in claim 19, further comprising semi-supervised machine learning for a model that is comprised of both unsupervised and supervised data sets whereby a person trains and tags data from a review of predicted data results of one or more data predictions.

22. The method disclosed in claim 16 or claim 19, further comprising a method for training supervised machined learning data by examining the results of a predicted health alert recommendation and transmitting the approval or correction to a large machine learning deep neural network algorithm; and using that new algorithm training for improved artificial intelligence model performance.

23. A system for automating medical care interactions, the system comprising:
a human process cognitive automation artificial intelligence model providing a patient with an automated interaction relating to medical and personal health information;
providing the patient with a communication based on the patient's medical data, the communication being either by text or verbal human sounding voice interactions, visual ques, sounds, or vibrations; and
the communication providing the patient with medical education, health recommendations, current medical analysis, health alerts, or a possible future medical event, wherein the cognitive automation is based on artificial intelligence generated from medical data belonging to more than one person.

24. The system disclosed in claim 23, further comprising a continuous artificial intelligence training feedback mechanism, wherein data collected is transferred to an artificial intelligence data analytics server to further improve the data analytics model, and continuously train a large machine learning deep neural network algorithm, wherein an application using the artificial intelligence algorithm is continuously updated to a newer and improved machine learning artificial intelligence model.

24. The system disclosed in claim 16 or claim 23, further comprising human process cognitive automation, wherein at least one interaction is initiated by a signal that is generated from data analyzed by artificial intelligence.

25. The system disclosed in claim 16 or claim 23, further comprising human interaction recommendations automated by at least one artificial intelligence algorithm based on at least one trigger from deep learning and artificial intelligence algorithms, including notifications, email, SMS, and artificial intelligence driven chatbot dialog interactions.

27. The method disclosed in claim 23, further comprising a scored interaction, wherein each interaction is weighted, including a dialog driven automated cognitive interaction that scores information gathered from an interaction, including from a questionnaire, wherein
the scoring mechanism determines whether an interaction is a non-concern, concern, or emergent concern; and
whether the interaction is classified as a compliant or non-compliant interaction.

28. The system disclosed in claim 16 or 23, further comprising a person's personal information, medical information, mental health information and wellbeing information to provide a cognitive interaction, which may include medical education and information to the patient for the purpose of collecting information for a machine learning model, a medical caretaker, and to provide therapeutic communications and interactions to the person.

29. The method disclosed in claim 16 or 23, further comprising feeding data back to the human process cognitive automation model, that can include provider supervised training.

30. The method disclosed in claim 16 or 23, further comprising permitting access to the patient's electronic medical record to at least one medical provider.

31. The method disclosed in claim 16 or 23, further comprising feeding data back to the human process cognitive automation model, that can include human supervised training.

32. The method disclosed in claim 16 or 23, further comprising permitting access to data by the patient to third parties, including family members, research studies, or medical personnel.

33. The method disclosed in claim 16 or 23, further comprising analysis of the patient's data by a human or artificial intelligence.

34. The method disclosed in claim 16, wherein the method of providing coordinated patient care is single use patient monitoring.

35. A method of operating the device disclosed in claim 1, wherein an observational mode is selected from the group comprising: single use mode, intermittent mode, or continuous mode, wherein
single use mode comprises obtaining one or more sensor readings at any desired point in time; intermittent mode comprises obtaining one or more sensor readings at a desired time interval over a fixed period of time, the selected sensors being predetermined prior to commencement of the observational period; and
a continuous mode comprising a continuous operation of one or more sensors for obtaining sensor readings over an observational period.

36. A method of operation of the device disclosed in claim 1, wherein an observational mode is selected from the group comprising: single use mode, intermittent mode, or continuous mode, wherein
single use mode comprises obtaining one or more sensor readings at any desired point in time; intermittent mode comprises obtaining one or more sensor readings at a desired time interval over a fixed period of time, the selected sensors being predetermined prior to commencement of the observational period; and
a continuous mode comprising a continuous operation of one or more sensors for obtaining sensor readings over an observational period, wherein data may be transmitted in real-time to a remote server for storage of the data in a patient data store.

37. A method of operation of the device disclosed in claim 1, wherein an observational mode is selected from the group comprising: single use mode, intermittent mode, or continuous mode, wherein
single use mode comprises obtaining one or more sensor readings at any desired point in time; intermittent mode comprises obtaining one or more sensor readings at a desired time interval over a fixed period of time, the selected sensors being predetermined prior to commencement of the observational period; and
a continuous mode comprising a continuous operation of one or more sensors for obtaining sensor readings over an observational period, wherein data is transmitted to a patient data store or a general data store, and shared with a permitted observer in real-time for remote patient monitoring, the permitted observer having obtained access to the patient data from a device administrator.

39. A method of operation of a medical device wherein the device can be operated remotely to obtain at least one biological sensor reading, the method comprising:
granting access to a remote operator by a device administrator;
granting permission to the remote operator to control the device, control of the device may include directing a sensor to obtain at least one biological sensor reading, or selecting a mode of operation for an observational period, other than single use mode;
granting permission to the remote operator for a specified period of time;
initiating a data transmission by the remote operator of at least one sensor reading; and
transmitting and storing data to the remote operator through a secure connection, wherein data is encrypted in transit and at rest.

40. A method of transmitting biological data from a device to a patient data store on a remote server, the method comprising:
obtaining data from a sensor on the device;
transferring data from the sensor to a network transceiver on the device;
transmitting data from the network transceiver to a remote server, using a secure and encrypted connection; and
storing the data in a patient data store.

41. A method of transmitting biological data from a device to an observer in real time, the method comprising:
adding an observer profile to a medical device data store;
granting permission to the observer to access historical and/or real-time data, wherein the permission granted may be ongoing or for a single session;
obtaining data from a sensor on the device;
transferring data from the sensor to a network transceiver on the device;
transmitting data from the network transceiver to a remote server, using a secure and encrypted connection; and
publishing the data to a real-time streaming data store and a historical data store, wherein the data can viewed by the observer from a computing device.

42. The method disclosed in claim 41, further comprising alerting the observer that there is an emergent medical event based on data analysis, algorithmic data analysis, or artificial intelligence analysis.

40. The method disclosed in claim 16, wherein artificial intelligence on the medical device is utilized to improve sensor accuracy.

41. The method disclosed in claim 16, wherein artificial intelligence on the medical device is utilized to predict at least one sensor reading.

42. A method of providing coordinated patient care by collecting, analyzing, and applying artificial intelligence to patient data, the method comprising:
obtaining patient medical data, including data obtained directly from a medical device, demographic patient data, behavioral health, mental health, and data found in a medical health record; and
transmitting the patient's data to a machine learning artificial intelligence data model;
wherein a person's data is applied against the machine learning artificial intelligence data model to generate conclusions based on the patient's information, such as patient summarization of medical information, recommendations of care, predictive heath outcomes, and cognitive automated patient interactions.

43. The method disclosed in claim 16, wherein data obtained directly from a medical device is used to continuously train and improve an artificial intelligence machine learning model used for predicting and analyzing patient data.

45. The method disclosed in claim 16, wherein patient data gathered from a medical device and/or a source other than the medical device is used to create a comprehensive patient data model for predicting and analyzing patient information.

51. The method disclosed in claim 41, further comprising observing more than one patient over a particular time period, wherein
more than one patient is identified for remote observation and has granted permission to the observer;
biological readings data, including audio and video data streams, are available to the observer in real time;
the user interface for the observer is a centralized observation dashboard for remote monitoring of patient data; and
alerting the observer that there is an emergent medical event based on data analysis, algorithmic data analysis, or artificial intelligence analysis.

52. The device disclosed in claim 1, further comprising conducting a health risk analysis utilizing algorithms and artificial intelligence to recognize an emergent event and automatically contact an emergency medical service and/or an enhanced 911 services; notifies the service of the emergent event; provides the GPS location of the device to the service; and transmits demographic information and data that is monitored and collected by the device, utilizing the network transceiver component of the invention.

53. A method of notifying a permitted third party of a medical event from a medical device, the method comprising:
initiating observation mode on the medical device;
identifying an emergent event utilizing sensor algorithms and artificial intelligence from one or more sensors; and
notifying the permitted third party of the likely emergent event.

54. The method disclosed in claim 53, further comprising transmitting a notification to an emergency medical service, identifying the likely medical event;
providing GPS location coordinates of the device to the emergency medical service;
delivering patient demographic information, such as the patient's name, address, and known medical information, including sensor reading data to the emergency medical service;
confirming receipt of the transmission; and
initiating a response by the emergency medical service.

53. The method disclosed in claim 19, further comprising:
accessing patient data in the patient-controlled patient data store, by a medical provider, wherein the medical device data can be transmitted and viewed by the medical provider in real-time;
conducting a virtual remote telehealth session between the patient and the medical provider, wherein the medical provider can assess the data in the patient data store during the telehealth visit to support a medical diagnosis and treatment plan; and
providing an update to the electronic medical record, which may include a transcript of the telehealth visit, notes regarding the visit, a medical order, and/or a diagnosis and treatment plan to the patient data store in real-time.

54. The method disclosed in claim 39, further comprising:
operating the medical device remotely to obtain one or more biological sensor readings in association with a virtual remote telehealth session, wherein:
the medical provider may assist the patient in taking a biological sensor reading remotely; and the medical provider may visually verify that the reading taken and the data transmitted to the provider is that of the patient associated with the virtual visit.

55. The method disclosed in claim 39, further comprising access to real-time and historical patient data from the medical device during a virtual remote telehealth session.

56. The method disclosed in claim 42, further comprising predicting a patient's current or future medical condition utilizing artificial intelligence directly on the device or a machine learning data analytics server.

57. The method disclosed in claim 23, further comprising:
a medical device with cognitive automation capabilities, including artificial intelligence programed on the device, that interacts with the patient directly to provide patient feedback, based upon medical information obtained from one or more sensors on the device or medical information recorded onto the device, to generate cognitive interactions directly on the device to elicit a positive, concerning, and/or alerting type communication to the patient.

58. The method disclosed in claim 23, further comprising:
a continuous artificial intelligence training feedback mechanism on a medical device where data collected on the device is transferred to an artificial intelligence data analytics server, consisting of data from interactions with the patient, the data collected on the device from patient interactions is transmitted to a data analytics server to further train and improve the data analytics model used on the device, wherein data consisting of more than one patient's information and is used to train a large machine learning deep neural network algorithm, wherein the artificial intelligence algorithm on the device is updated to a newer and improved machine learning artificial intelligence model.

60. The method disclosed in claim 23, further comprising:
providing health feedback to the patient regarding the patient's medical status, based on one or more patient informational data streams, including data from a medical device; and
initiating a communication to the patient regarding a change to the patient's medical status.

61. A method of data transmission for real time medical biological readings obtained directly from a medical device, the method comprising:
connecting a medical device to the internet through a network transceiver on the medical device, the transceiver protocols on the medical device provide for continuous publishing and subscribing of biological medical data;
transmitting the biological medical data from the medical device network transceiver to a receiving device through an internet connection.

62. The method disclosed in claim 61, wherein biological medical data is transmitted from a medical device to a receiving device through a peer-to-peer connection, the method further comprising:
selecting the data recipient on the medical device, the device initiating a biological data reading,
and transfers data through a secured peer-to-peer connection; and
connecting the receiving device to the medical device through a secured connection that transmits data in real-time.

63. The method disclosed in claim 61, wherein biological medical data is transmitted from a medical device to a receiving device through an intermediate server, the method further comprising:
selecting the data recipient on the medical device, the device initiating a biological data reading, and transfers data through a secured connection to an intermediate server; and connecting the receiving device to the routing server through a secured connection that transmits data in real-time.

64. A method for synchronizing data on a receiving device, the method comprising:
collecting data on a medical device;
choosing a remote data store or a receiving device data store, on the medical device;
upon establishing internet connectivity, sending data not previously transmitted directly to the remoted data store or receiving device data store; and
synching the data on the remote data store or receiving device data store with data on the medical device.
